(19) United States
(12) Patent Application Publication
(10) Pub. No.: US 2021/0113736 A1
(43) Pub. Date: Apr. 22, 2021

(54) OXYGEN-RELEASING BIOMATERIALS, ARTICLES AND METHODS

(71) Applicant: University of Massachusetts, Boston, MA (US)

(72) Inventors: Gulden Camci-Unal, Lowell, MA (US); Darlin Antigua, Lowell, MA (US); Sanika Suvarnapathaki, Lowell, MA (US)

(21) Appl. No.: 17/073,332

(22) Filed: Oct. 17, 2020

Related U.S. Application Data
(60) Provisional application No. 62/916,320, filed on Oct. 17, 2019.

(51) Int. Cl. A61L 27/18; A61K 9/06; A61K 47/34; A61K 47/42; A61K 33/40; A61L 27/54; A61L 27/58; A61L 27/52; A61L 27/44; A61L 27/22; A61L 27/26; C12N 5/00

(52) U.S. Cl. CPC A61L 27/18; A61L 2430/20; A61K 47/34; A61K 47/42; A61K 33/40; A61L 27/54; A61L 27/58; A61L 27/52; A61L 27/446; A61L 27/222; A61L 27/26; C12N 5/0068; A61L 2430/40; A61L 2400/18; A61L 2300/11; C12N 2533/54; C12N 2533/40; A61K 9/06

(57) ABSTRACT

An oxygen-releasing biomaterial, articles and methods, and more particularly an oxygen-releasing biomaterial with sustained oxygen-releasing properties of four to five weeks, which is suitable for tissue engineering scaffolds, is disclosed. The biomaterial contains a hydrogel with a plurality of microparticles suspended in the hydrogel. The microparticles contain an oxygen carrier that is encapsulated in a biocompatible hydrophobic material, where the release of oxygen from the oxygen carrier is sustained over a four to five week period. The biomaterial has application in tissue engineering, osteogenesis, burn and wound treatment, and treatment of cardiac conditions, and has further antimicrobial properties.

### CROSS-REFERENCE TO RELATED APPLICATION

This application claims priority to earlier filed U.S. Provisional Application Ser. No. 62/916,320, filed Oct. 17, 2019, the contents of which are incorporated herein by reference.

### BACKGROUND OF THE INVENTION

### 1. Field of the Invention

The present patent document relates generally to oxygen-releasing biomaterials, articles and methods, and more particularly to an oxygen-releasing biomaterial with sustained oxygen-releasing properties of at least four to five weeks.

### 2. Background of the Related Art

Tissue engineering has transformed the existing biomedical strategies that are aimed to address the need of organ transplants. While progress has been achieved, the clinical need for organ transplants and successful alternatives for patients persists. As of March 2020, there are 112,568 patients that have been placed on the organ transplant waiting list in the United States. Yet, only 39,718 of these patients were removed from this waiting list, as reported by the Health Resources & Services Administration (OPTN/SRTR Annual Report). Biomaterials and tissue engineering have evolved and shown alternative clinical approaches that can support organ-scaled tissue constructs for these patients

Oxygen supply is essential for the long-term viability and function of tissue engineered constructs in vitro and in vivo. For tissue constructs in vivo, the host blood supply serves as the primary source of oxygen to the encapsulated cells. The integration with the host blood supply occurs over the course of 4 to 5 weeks and involves neovascularization stages to support the delivery of oxygenated blood to the tissue construct. During this process, the cells encapsulated within the tissue construct are prone to oxygen deprivation, cellular dysfunction, damage, and hypoxia-induced necrosis. The success of in vivo tissue constructs relies on the integration with the host vasculature. The vascular integration of these constructs is responsible for delivering growth factors, cell signals, nutrients, and most critically oxygen. This oxygen supply is essential for cell viability, proliferation, and function. The first 4 to 6 weeks post-implantation of a tissue construct includes the tissue remodeling stages involved in wound healing and neovascularization. The absence of vasculature during this time can halt cell growth and repair, as well as lead to further tissue damage by hypoxia-induced necrosis. Moreover, the cells within tissue constructs often become oxygen deficient the scaffold dimensions are greater than 100 μm to 200 μm due to diffusion limitations to oxygen. Recent strategies to prevent these issues have involved treating cell samples and co-encapsulating cells in scaffolds with growth factors that promote vascularization, such as vascular endothelial growth factor (VEGF). The caveat of these methods is that it may lead to a non-homogenous distribution throughout a three-dimensional (3D) biomaterial scaffold. There are also microfluidic approaches that hold promise for neovascularization in vitro; however, the shortcomings of these techniques are due to the limitations to the level of resolution achieved and troubleshooting required, depending on the microfabrication technique utilized. It is possible to use these methods in combination to work synergistically; however, the lack of source of oxygen until optimum homogeneous vascularization can be established which typically takes 4-5 weeks, remains an issue during tissue remodeling and vascularization stages regardless.

17.9 million lives are claimed annually by Cardiovascular diseases alone which amount to 31% of deaths worldwide. Over 20 million patients suffer from tissue loss relevant to cardiovascular ailments. For such cardiovascular disorders tissue engineering strategies provide an effective solution. Specifically, myocardial infarction and ischemic heart disease are key contributors to high mortality with few available treatment options. Most therapies delay progression of the heart disease and usually involve immune suppressive components following the highly invasive procedure. The alternative option in many cases is cardiac transplantation, which depends largely on organ donor availability and compatibility. The long-term clinical success of the organ transplant is determined by whether the donor organ is accepted by the patient's body. In cardiac tissue engineering, the conventional solutions to address this need for donor organs have created a great scientific impetus to improve tissue engineering strategies to develop three-dimensional (3D) organ scale cardiac tissue constructs. Efforts to engineer cardiac tissue constructs utilize biodegradable polymers for developing in vitro scaffolds for delivery of cardiac cells for tissue regeneration. While vast array of biomaterials have been explored by existing studies, many of these biomaterials are limited in their functional capabilities to support long-term cell viability and metabolic activity.

Maintaining a high cell viability is a major challenge in 3D organ scale tissue engineering because of diffusion limitations for oxygen and nutrients beyond the 300 μm range. Particularly, cardiac tissue is a high oxygen demand tissue which can consume up to 70 ml O₂/min/100 g oxygen during strenuous activity. Therefore, in cardiac tissue regeneration, oxygen availability is vital facilitate optimum growth and function and prevent hypoxia induced necrosis. Host blood supply is the primary source of oxygen and nutrients to the encapsulated cells in the engineered tissue construct. Upon in vivo implantation, it can take up to four weeks for them to integrate with the host's vasculature. Therefore, there has been an increased focus on developing strategies to improve vascularization of tissue constructs. However, improving vascularization alone does not address the lack of immediate oxygen needed for maintaining cell viability and function to ensure the clinical success of the tissue-engineered constructs. Therefore, efforts to develop biomaterials that themselves release oxygen and provide it immediately within the cellular microenvironment has been largely explored as a possible solution until optimum vascularization can occur.

Oxygen-diffusion limitation is one of the primary reasons for low cell viability in tissue-engineered constructs. Specifically, organ scale constructs larger than 300 μm are challenged by diffusion limitations for oxygen and nutrients which are vital to their in vivo success.

To date, research has shown the ability of oxygen-releasing biomaterials to release oxygen for only for short period of times.

According to the National Institutes of Health (NIH), 1.9 million cases project annually of patients who will acquire cranial fracture. Among these common cases are pediatric patients of which a range of 2%-20% of the pediatric head trauma cases will result in cranial damage. These critical orthopedic defects are clinically remedied through bone autografts and allografts. Regardless, the clinical discrepancy continues between the number of patients who suffered a critical bone injury and number bone substitutes available for patients. The field of bone tissue engineering is motivated to expand the number of bone substitutes, as well as advance the state-of-the-art techniques that support osteogenesis and bone remodeling. Importantly, research in this area aims to preserve the myriad of intrinsic functions of bone after trauma. Bone mechanically supports and protects internal organs and tissues while also providing storage units for inorganic minerals such as calcium. The complex and organized microenvironment of bone is essential for vital physiological processes such as hematopoiesis. Therefore, the research efforts to define and understand the biomaterial properties and parameters that control bone regeneration such as osteoconductive and osteoinductive behaviors have been increasingly sought.

There has been a tremendous need to develop bone substitutes that are capable of providing clinical success. As of 2016, the GlobalData estimates a $2.6 billion global market for bone remodeling biomaterials, such as bone grafts and synthetic substitutes. By 2023, this market is forecasted to reach $3.3 billion across 49 international markets. While there has been immense progress, autografts remain as the gold standard for grafting material in bone substitutes due to their superior osteoinductive and osteoconductive behavior, and low immunogenicity. The caveat has been the limited availability of these constructs as well as potential clinical risks such as donor site morbidity during the procedures. The alternative option is allografts which are more accessible, but harbor possible risks such as potential disease transmission and immunological rejection.

Available commercial products currently sold in the market are unable to effectively heal second and third degree burn wounds in resource-limited settings. In addition, these products cannot provide effective wound healing to devastating burn injuries. Therefore, we have developed a hydrogel-based dressing formulation that is sprayable and rapidly cure on second and third degree burn wounds of patients.

### SUMMARY OF THE INVENTION

These challenges have prompted the innovation of biomaterials that can release oxygen. In this patent document, calcium peroxide (CaO₂) in combination with polycaprolactone (PCL), a hydrophobic biopolymer, to produce scaffolds that provide sustained oxygen release over extended tissue culture periods is demonstrated. These oxygen-generating scaffolds support the survival, proliferation, and function of diverse cell types encapsulated in three-dimensions (3D) and under induced hypoxia. The broad basis of this work supports prospects in the expansion of robust and clinically translatable tissue constructs. Thus, some oxygen-releasing biomaterials have emerged to achieve homogenous results in organ-scale tissue constructs, which develops beyond wound healing stages. The common materials used for this purpose include solid peroxides, liquid peroxides, and fluorinated compounds as oxygen carriers. However, solid peroxide such as calcium peroxide (CaO₂) is preferred for its high yield of pure oxygen and low toxicity. The hydrolysis of this compound generates oxygen as the byproduct of the reaction as shown in the proceeding chemical equation:

CaO₂+2H₂O→Ca(OH)₂+H₂O₂

2H₂O₂→2H₂O Equation:

Solid peroxides can introduce potential risk for uncontrollable burst release of oxygen during hydrolysis that is damaging to surrounding tissues in vitro and in vivo. A pioneering strategy to control the release of oxygen and offset this effect is to limit the rate of exposure to the water content in the cellular microenvironment by using a hydrophobic barrier. The advantage of using this approach is it offers a facile method to modify a tissue culture system to support a three-dimensional (3D), organ-sized construct during its integration in the host.

In one embodiment, the synthesis, and material characterization of novel oxygen-generating scaffolds consisting of oxygen generating microparticle-reinforced gelatin hydrogel is disclosed. Specifically, these microparticles include a hydrophobic material, such as polycaprolactone (PCL) to encapsulate calcium peroxide (CaO₂) as a source for oxygen release. The effect of modifying this hydrophobic barrier on the oxygen release of these scaffolds is explored. The scaffolds are extensively assessed for their mechanical behavior, cytocompatibility, and cytotoxicity. Oxygen release kinetics and biological performance in long-term cell cultures in vitro with different cell types 3D-encapsulated within the scaffold is also explored.

The hydrolysis kinetics involved in the breakdown of a solid peroxide, such as calcium peroxide, can be manipulated to control the amount of oxygen-release. A novel strategy to reduce the contact of the solid peroxide with water in the surroundings is the incorporation of a hydrophobic barrier around it. In one embodiment, calcium peroxide (CaO₂) was encapsulated into polycaprolactone (PCL) to achieve a gradual generation and release of oxygen. Herein, a scaffold that is reinforced with microparticles, composed of calcium peroxide and PCL is described. These oxygen-generating scaffolds were characterized in vitro for their biological, chemical, and mechanical effects on the cell viability, cellular functions, and in vitro osteogenic differentiation.

In one embodiment, a novel oxygen-releasing biomaterial for improved viability, growth, and metabolic activity of H9c2 cardiac cells that surpasses the oxygen-releasing capabilities of modern biomaterials by showing controlled sustained oxygen release for up to 4 weeks and lasting oxygen levels up to 5 weeks. Through the use of calcium peroxide (CaO2) as an oxygen source, along with polycaprolactone (PCL) as a hydrophobic polymer, oxygen generating microspheres were developed using an emulsification technique by using CaO2 which was encapsulated within hydrophobic PCL. These composite CaO2-PCL oxygen generating microspheres were co-encapsulated with H9c2 cardiomyocytes within a gelatin methacrylate matrix (GelMA), to form the oxygen generating scaffolds and were cultured under hypoxia to mimic the physiological in vivo environment.

In one embodiment, these oxygen-generating scaffolds are emerging biomaterials to support osteogenesis and provide continuous oxygen supply as they integrate into the injury site. In addition to oxygen-release, the porosity and bioactivity of these scaffolds also support osteogenesis. The oxygen-generating scaffolds incorporate oxygen-generating compounds, such as solid peroxides, liquid peroxides, or fluorocarbons which act as oxygen carriers, are encapsulated in a hydrophobic material, such as a biocompatible and biodegradable plastic, such as PCL.

In one embodiment, a wound dressing contains a component with high-oxygen content to provide antimicrobial properties and deliver oxygen to improve healing. The wound dressing may contain an anti-inflammatory component to aid and accelerate wound healing. In one embodiment, a collagen-based matrix acts as a bioactive component, hyaluronic acid may be included as an anti-inflammatory component, and peroxide-encapsulated polycaprolactone microparticles as the antimicrobial component. The wound dressing may be formulated as a sprayable hydrogel dressing for the pre-treatment of deep dermal and full thickness wounds. It has quick and easy administration features, allows for on-site management of the burn wound rapidly, maintains a physical barrier, is adhesive, and non-toxic to the tissue. The sprayable dressing formulation is intended to remain intact up to 3 weeks covering the wound from the time of the injury until an appropriate care unit can surgically treat the patient if necessary.

In another embodiment, a novel sprayable hydrogel-based wound dressing that can easily be applied on the burn wounds in prehospital settings. In addition, the invention may provide appropriate burn care closer to the point of injury and therefore allow for better long-term outcomes. The proposed dressing formulation will make an original contribution and lead to improved outcomes in healing of large burn wounds. This invention will accelerate progress in medical research for patients with reconstructive and rehabilitative needs after traumatic burn injuries.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and other features, aspects, and advantages of the present invention will become better understood with reference to the following description, appended claims, and accompanying drawings where:

FIG. 1 shows a table of nomenclature for oxygen-generating scaffolds disclosed herein (Table 1);

FIG. 2 shows at inset (A) an in vitro pig skin model to demonstrate the application process of the wound dressing, where the formulation contains only 5% (w/v) GelMA without peroxide-encapsulated polycaprolactone microparticles via a spray bottle, at inset (B) a 4 cm by 4 cm sized pig skin model before dressing application, at inset (C) a sprayed wound dressing on pig skin, at inset (D) UV light exposure for material solidification, and at inset (E) the formation of crosslinked hydrogel covering the pig skin model;

FIG. 3 shows at inset preliminary data for a sprayable antimicrobial wound dressing formulation containing 10% (v/w) GelMA with microparticles containing an oxygen carrier (60 mg/mL CaO2 in PCL) on an in vitro porcine skin model, where inset (A) shows 4 cm by 4 cm sized pig skin model, inset (B) shows formation of crosslinked hydrogel covering the pig skin model, inset (C) shows microscopic image of microparticles on the porcine skin, and inset (D) shows a phase contrast microscopic image of the microparticles on porcine skin;

FIG. 4 shows synthesis and characterization of microparticles having an oxygen carrier where SEM images of the microparticles are shown at insets (a), (b), and (c), swelling properties at inset (d), degradation properties at inset (e), and mechanical properties tested by DMA compression test for Pristine GelMA at inset (f), where the date represents 0CPO, 40CPO, and 60CPO scaffolds which contain 0 mg, 5.4 mg, and 8.1 mg net CaO2 in the GelMA matrix, respectively;

FIG. 5 shows cellular response to the oxygen generating scaffolds, for Alamar Blue assay for evaluation of metabolic activity of (a) 3T3 Fibroblasts, (b) L6 rat myoblasts and (c) Cardiac fibroblasts; Lactate Dehydrogenase (LDH) assay for evaluation of cytotoxicity of (d) 3T3 Fibroblasts, (e) L6 rat myoblasts and (f) cardiac fibroblasts; Caspase Glow 3/7 assay for evaluation of cellular apoptosis of (g) 3T3 Fibroblasts, (h) L6 rat myoblasts and (i) Cardiac fibroblasts, where the data represented is for the Pristine GelMA, 0CPO, 40CPO, and 60CPO scaffolds which contain 0 mg, 5.4 mg, and 8.1 mg net CaO2 in the GelMA matrix, respectively;

FIGS. 6(a)-6(d) show release kinetics of oxygen generating scaffolds cultured under hypoxia with catalase in media 6(a) without cells microencapsulated, 6(b) with 3T3 fibroblasts microencapsulated, 6(c) with L6 rat myoblasts microencapsulated, and 6(d) with primary cardiac fibroblasts microencapsulated;

FIG. 7 shows cumulative oxygen release measured as a result of change in PCL concentration;

FIG. 8 shows the effect of oxygen generating scaffolds on pH of the culture media as observed on days 1, 4, 7, 14, 21, and 35 when cultured (a) without cells under hypoxia and (b) with primary cardiac fibroblasts microencapsulated, where the data is represented for the Pristine GelMA, 0CPO, 40CPO, and 60CPO scaffolds which contain 0 mg, 5.4 mg, and 8.1 mg net CaO2 in the GelMA matrix, respectively;

FIG. 9 shows characterization of physical properties of oxygen-releasing biomaterial performed by (a) phase contrast microscope image, (b) SEM analysis, and (c) SEM image to characterize interaction with the hydrogel matrix, (d) evaluating mean particle diameters and size distribution per batch, (e) DMA compression test, (f) swelling analysis and (g) degradation analysis;

FIGS. 10(a)-10(d) shows oxygen release kinetics of different scaffold compositions with microencapsulated H9c2 cardiomyocytes evaluated using the NeoFox oxygen sensing probe under normoxia 10(a) without catalase, 10(b) with catalase, and under hypoxia 10(c) without catalase, and 10(d) with catalase;

FIG. 11 shows cellular metabolic activity of encapsulated H9c2 Cardiomyocytes evaluated using Alamar Blue assay under normoxia (a) without catalase and (b) with catalase, and under hypoxia (c) without catalase and (d) with catalase;

FIG. 12 shows cytotoxicity evaluation for the oxygen generating scaffolds using Lactate Dehydrogenase (LDH) assay for scaffolds that encapsulated H9c2 Cardiomyocytes cultured under normoxia (a) without catalase and (b) with catalase, and under hypoxia (c) without catalase and (d) with catalase;

FIG. 13 shows apoptosis response to oxygen generating scaffolds evaluated using Caspase Glo 3/7 assay for scaffolds that encapsulated H9c2 Cardiomyocytes cultured under normoxia (a) without catalase and (b) with catalase, and under hypoxia (c) without catalase and (d) with catalase;

FIG. 14 shows evaluation and monitoring of pH changes in media exposed to and used for culturing oxygen generating scaffolds that encapsulated H9c2 Cardiomyocytes with different CaO2 concentrations cultured under normoxia (a) without catalase and (b) with catalase, and under hypoxia (c) without catalase and (d) with catalase;

FIG. 15 shows synthesis and characterization of oxygen generating scaffolds for tissue regeneration where insets (a), (b), and (c) show SEM images of the oxygen generation scaffolds, inset (d) shows swelling ratios of oxygen generating scaffolds cultured in media, and inset (e) shows degradation;

FIG. 16 shows measurement of oxygen-release kinetics in vitro for scaffolds that encapsulated preosteoblasts cultured under hypoxia with catalase in media (a) without cells and (b) with 3D-encapsulated preosteoblasts;

FIG. 17 shows evaluation of cellular response to the oxygen-generating scaffolds that encapsulated preosteoblasts (a) metabolic activity evaluation using Alamar Blue assay and (b) Alkaline phosphatase activity evaluation using the ALP assay; and

FIG. 18 shows evaluation of pH, biocompatibility, cellular apoptosis for preosteoblasts in vitro through (a) pH measurements, (b) LDH cytotoxicity assay, and (c) Caspase Glo 3/7 assay;

FIG. 19 shows gene expression of preosteoblasts microencapsulated in oxygen-generating scaffolds for (a) Osteoclacin (OCN) expression and (b) Bone morphogenic protein (BMP-7) gene expression; and

### DESCRIPTION OF THE PREFERRED EMBODIMENT

This patent document discloses oxygen-releasing biomaterials, articles and methods, and more particularly an oxygen-releasing biomaterial with sustained oxygen-releasing properties of four to five weeks, which is suitable for tissue engineering scaffolds. Most oxygen-releasing biomaterials show a sudden release of oxygen due to poor control over the hydrolysis reaction rate, which may damage cells. Therefore, sustained oxygen-release capabilities for tissue engineering applications is essential for biomaterials. The release kinetics of an ideal oxygen-releasing biopolymer should be tunable and extended from days up to weeks to allow sufficient time for revascularization and maturation of the engineered graft within the host system. The oxygen-releasing micro-particles are fabricated by encapsulating a solid peroxide, such as solid peroxides, liquid peroxides, or fluorocarbons, inside a hydrophobic material made from a biocompatible plastic, such as poly dimethyl siloxane, polylactic co-glycolic acid, or poly vinyl pyrrolidone. Calcium peroxide (CaO2) has been used by way of example and not limitation in these examples as it is suitable for use in the human body. Similarly, a polycaprolactone (PCL) has been used by way of example and not limitation as the hydrophobic material as it is biocompatible. An emulsion-based fabrication technique may be used. Incorporation of oxygen releasing micro-particles using a hydrophobic material, helps to achieve a sustained oxygen release over a four to five week period. The use of a hydrophobic material slows down the rate at which the water from the encapsulating hydrogel reacts with the encapsulated oxygen carrier, such as CaO₂. An advantage of this approach is the slow release of oxygen allows for production of sufficient oxygen to improve and sustain a high cell viability and metabolic activity for the cardiac myocytes and primary cardiac fibroblasts which are high oxygen demanding cells. The present method also has the advantage of the ability to deliver oxygen on demand in a controlled manner over an extended period of time. The biomaterial disclosed herein also has the advantages of being biocompatible and biodegradable. Further, the use of microparticles can potentially enable fabrication of injectable delivery to the necrotic tissue site and eliminate the need for invasive surgeries to implant the oxygen releasing biomaterials. These microparticles may range in size from 50 μm to 250 μm, with average microparticle size of 100 μm.

In one embodiment, an oxygen-releasing biomaterial is disclosed. The biomaterial contains a hydrogel with a plurality of microparticles suspended in the hydrogel. The microparticles contain an oxygen carrier that is encapsulated in a biocompatible hydrophobic material, where the release of oxygen from the oxygen carrier is sustained over a four to five-week period. In one embodiment, the oxygen carrier comprises 5-25% w/v of the hydrophobic material. In another embodiment, the oxygen carrier comprises 13.5% w/v of the hydrophobic material.

In one embodiment, a method of osteogenesis is disclosed where an oxygen-releasing biomaterial including a hydrogel and a plurality of microparticles suspended in the hydrogel is provided. The microparticles comprising an oxygen carrier are encapsulated in a biocompatible hydrophobic polymer. The oxygen releasing biomaterial is applied to the damaged bone tissue.

In one embodiment, a method of providing oxygen-releasing biomaterial to improve vascularization of damaged cardiac tissue is disclosed. An oxygen-releasing biomaterial including a hydrogel and a plurality of microparticles suspended in the hydrogel is provided. The microparticles comprising an oxygen carrier are encapsulated in a biocompatible hydrophobic polymer. The oxygen releasing biomaterial is applied to the damaged cardiac tissue.

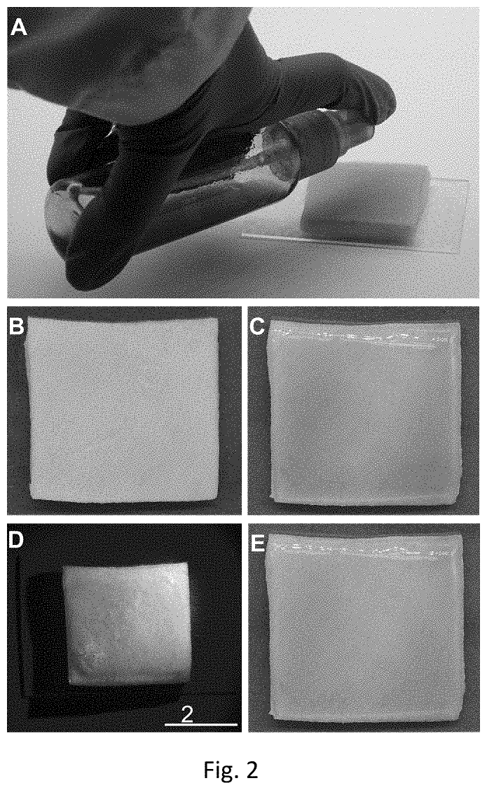

In one embodiment, a wound dressing in a sprayable form is disclosed. FIG. 2 shows at inset (a) an in vitro pig skin model to demonstrate the application process of the wound dressing, where the formulation contains 5% (w/v) of hydrogel (e.g. GelMA) without peroxide-encapsulated polycaprolactone microparticles via a spray bottle. However, in another embodiment, the formulation may contain 3-20% (w/v) of hydrogel. At FIG. 2, inset (b), a 4 cm by 4 cm sized pig skin model before dressing application. At FIG. 2, inset (c), a sprayed wound dressing on pig skin is shown. At FIG. 2, inset (d), a UV light exposure for material solidification of the hydrogel. At FIG. 2, inset (e), the formation of crosslinked hydrogel covering the pig skin model. The application of the sprayable wound dressing formulation showed to be a rapid process (180 secs) providing full covering of the sprayed area and showed no sign of material disintegration after the application process was completed.

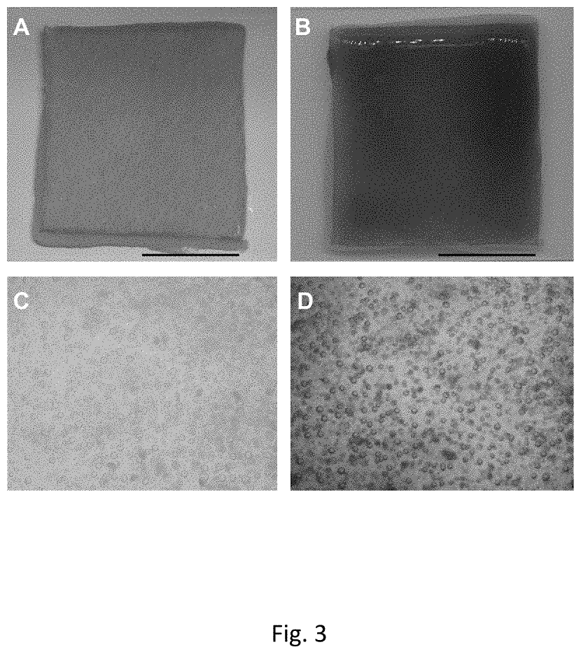

In one embodiment a sprayable wound dressing is shown at FIG. 3, with antimicrobial properties. The sprayable antimicrobial wound dressing formulation may contain 10% (v/w) of hydrogel (e.g. GelMA), with oxygen releasing microparticles (60 mg/mL CaO2 in PCL) on an in vitro pig skin model. However, in another embodiment, the formulation may contain 3-20% (w/v) of hydrogel. FIG. 3, inset (A) shows a 4 cm by 4 cm sized pig skin model. FIG. 3, inset (B), shows formation of crosslinked hydrogel covering the pig skin model. FIG. 3, inset (C) shows microscopic image of oxygen releasing microparticles on the pig skin. FIG. 3, inset (D), shows phase contrast microscopic image of oxygen releasing articles on porcine skin. The application of the sprayable wound dressing formulation showed to be rapid (180 secs) providing full covering of the sprayed area and complete homogenous distribution of peroxide-encapsulated polycaprolactone microparticles on the model pig skin. The application of the sprayable wound dressing formulation may provide fill covering that varies between 5-200 secs.

### EXAMPLES

The present disclosure will be described in greater detail by way of the following specific examples. The following examples are offered for illustrative purposes, and are not intended to limit the invention in any manner. Those of skill in the art will readily recognize a variety of non-critical parameters that can be changed or modified to yield alternative embodiments according the invention.

### Example 1—Oxygen Generating Biomaterials

### Materials

Polycaprolactone (PCL) pellets were acquired from Capa, Fischer Scientific. Calcium peroxide (CaO₂) was supplied by Sigma Aldrich. Porcine Skin Gelatin 100 g was purchased from Sigma Aldrich. Methacrylic anhydride was obtained from Sigma-Aldrich (St. Louis, Mo.). Dulbecco's phosphate buffered saline (DPBS), Dulbecco's Modified Eagle's Medium (DMEM—low glucose), fetal bovine serum (FBS), trypsin-ethylenediaminetetraacetic acid (EDTA) 0.25%, and penicillin/streptomycin (P/S) were purchased from Gibco (Thermo Fisher Scientific, Inc., Waltham, Mass.). Alamar Blue reagent was obtained from Invitrogen (Grand Island, N.Y.). 2-hydroxy-1-[4-(hydroxyethoxy) phenyl]-2-methyl-1propanone (Irgacure 2959) was acquired from BASF Corporation (Florham Park, N.J.). Lactate Dehydrogenase (LDH) activity kit was obtained from Genesee Scientific. Caspase glo 3/7 assay kit was secured from Promega. NeoFox Oxygen sensing probe was procured from Ocean Optics Inc. All reagents were used as received without further purification.

### Synthesis of GelMA

The hydrogel component of the oxygen-generating scaffolds contained 5% (w/v) porcine GelMA (GelMA), and 0.5% (w/v) Irgacure 2959 as the photoinitiator component. For this precursor solution, 10 g of porcine skin gelatin was dissolved in 100 mL of DPBS at 50° C. Then, methacrylic anhydride (MAA) was added dropwise to the stirring gelatin solution. After a total of 8 mL of MAA was added, the mixture continues to stir for 4 hours at 50° C. and 200 rpm. This reaction has been optimized for the methacrylation of the gelatin backbone. The methacrylation reaction was quenched with the addition of 300 mL of DPBS into mixture. Using nitrocellulose membranes, the methacrylated gelatin is dialyzed and submerged in distilled water for one week under constant magnetic stirring (180 rpm) at 40° C. The dialyzed solution was subsequently stored overnight at −80° C. The solution was lyophilized for one week to obtain the GelMA polymer in a foam form for use.

### Synthesis of Oxygen-Releasing Microparticle

The oxygen-generating microparticles are synthesized by encapsulating CaO₂ within a hydrophobic phase, PCL. The hydrophobic phase of this material is prepared by dissolving PCL in chloroform to a 13.5% (w/v) solution; this step occurred under constant stirring conditions and at room temperature. Then, CaO₂ is added to the PCL solution at varying concentrations of either 0, 40, or 60 mg/mL. Based on the concentrations, the microparticle content was hypothesized to produce distinct oxygen-release profiles. Following, the CaO₂ and PCL solution continues to stir form a complex. The aqueous phase used in the emulsification process consisted of a low molecular weight polyvinyl alcohol (PVA) dissolved in deionized water at 80° C. to form a 0.5% (w/v) solution. During the microparticle fabrication process, the PVA solution is the aqueous phase while the PCL-CaO₂ solution was introduced to act as the inner viscous phase. Subsequently, the PCL solution was added dropwise to the PVA solution under constant magnetic stirring. These microparticles were transferred into conical tubes and centrifuged at 800 rpm. This process allows the aqueous and hydrophobic phases to separate in layers, with the aqueous layer at the top. The supernatant containing the PVA solution was decanted and the particles are washed with chloroform three times to remove residual PVA. These oxygen-releasing microparticles were vacuumed dried for 4 hours to evaporate residual chloroform.

### Fabrication of Oxygen-Generating Scaffolds

The scaffolds composed of GelMA, and were reinforced with the synthesized oxygen-releasing microparticles. For the precursor solution, the microparticles are homogenously mixed into the GelMA prepolymer solution at 0, 40, and 60 mg/mL concentrations. After the microparticles are added in GelMA, the precursor solution is pipetted at the bottom of 96-well plate at volume of 40 μL. The precursor solution is UV-crosslinked (Omnicure 52000 (EXFO Photonic Solutions Inc., Ontario, Canada) at 700 mW/cm². The crosslinking times were optimized in proportion to the CaO₂ concentration within the scaffolds. For material characterization studies, scaled versions of hydrogels at 100 μL in volume were utilized. The prepolymer solution is also photocrosslinked similarly in these larger hydrogels. However, the precursor solution is then pipetted in between a 1 mm thick glass spacer rather than a well plate. This prepolymer form is UV-crosslinked for 20, 40, 60, and 80, seconds at a power of 700 mW/cm² for the Pristine GelMA, 0CPO, 40CPO, and 60CPO gel conditions, respectively. These various microparticle-reinforced scaffolds are then submerged in PBS and stored until use.

### Swelling and Degradation Analysis

The swelling and degradation behavior of the oxygen-releasing scaffolds were analyzed using the larger-scaled hydrogels at 100 μL in volume. The scaffolds for swelling analysis were stored in DPBS for 48 hours for equilibrium swelling, with four replicates per each gel composition. Then, the gels were removed from the medium and the residual DPBS on the gel was absorbed using a Kimwipe. Each scaffold samples was weighed and transferred into Eppendorf tubes to be stored in −80° C. for 24 hours. Following, these samples were lyophilized to obtain the dry weight of the material. The swelling ratio of the gel samples were determined by dividing the wet weight after equilibrium swelling by their corresponding dry weights. The resulting ratios are reported as percentage values.

The degradation behavior of these oxygen-generating scaffolds was evaluated using an enzymatic degradation experiment. Specifically, collagenase type II was utilized as a physiological enzyme to facilitate degradation. The samples were prepared as previously described for swelling experiments. The fabricated gels were transferred into weighed Eppendorf tubes and stored at −80° C. overnight. These samples were also lyophilized for an additional 24 hours to dehydrate the material. The initial dry weight is determined by subtracting the weight of the empty Eppendorf tube from the total weight of the sample and its respective container. Following this, these samples were rehydrated by submerging the gels in 1 mL of PBS for 24 hours. The PBS was subsequently removed and replaced with 1 mL of 2.5 U/mL of collagenase type II in PBS. To initiate the enzymatic reaction the samples were incubated at 37° C. on a shaker. The degradation behavior was monitored by measuring the mass remaining at various time points (3, 6, 12, 18, 24, 36, and 48 hours). At these time points, the samples were rinsed with PBS to wash enzymatic content completely and prevent further degradation of the material. Then, these samples are stored overnight at −80° C. and lyophilized the following day to obtain the dry weight. This process is repeated until the final dry weight is determined at 48 hours. We utilized four replicates per each scaffold composition. The degradation behavior is reported as the percent mass remaining after degradation. For each time point, this value was determined by dividing the dry weight after degradation by the initial weight of the hydrogel. The percent (%) mass remaining at the end of the last time point represents the total degradation over 48 hours.

### Mechanical Testing

The mechanical performance of these oxygen-generating scaffolds was examined through compression tests using Dynamic Mechanical Analysis (DMA). The scaled scaffold samples were also used in this physical characterization. These samples swelled in PBS for 24 hours and were shaped using an 8 mm biopsy punch prior to compression tests. Following, the samples were removed from the PBS and excess moisture on the material was removed using a Kimwipe. The controlled parameters set during mechanical testing utilized a preload force of 0.0010 N, an isothermal temperature of 23° C., a soak time of 1 minute, force ramp rate of 0.1 N/min, and an upper force limit of 2 N. The data yielded a stress-strain curve of which the slope of the linear region was utilized to determine the compressive modulus of each scaffold composition.

### Porosity

The microarchitecture of the synthesized oxygen-generating microparticles and the pore size of the scaffolds were evaluated under a scanning electron microscope (JEOL 5200 SEM). The scaffolds samples for SEM imaging were flash frozen in liquid nitrogen and lyophilized before being placed in argon atmosphere for gold coating. This work used the software ImageJ by the National Institutes of Health (NIH) to measure the percent porosity and pore size and distribution for all microparticle and scaffold compositions. The SEM images also revealed the morphological characteristics of the PCL-CaO₂ microparticles interfacing with the GelMA matrix in the scaffold.

### Oxygen-Release Profiles

The oxygen release profiles of the synthesized scaffolds were investigated in hypoxic cell culture conditions. This work utilized the NeoFox optical oxygen sensing probe to measure the percent (%) dissolved oxygen. The optical oxygen sensing probe was submerged in the scaffolds with and without cells 3D encapsulated. The induced hypoxia was controlled in a hypoxia chamber (StemCell Technologies). In this in vitro environment, the cells are limited to oxygen-generating microparticles as the primary source of oxygen. The effect of catalase on oxygen levels was observed by adding catalase at 1 mg/mL to the culture media in a separate condition. The enzyme, catalase, has been shown to support and improve the conversion efficiency from hydrogen peroxide to water and oxygen during hydrolysis. Therefore, we included the catalase condition to determine if the presence of catalase can improve oxygen-release in in vitro cell culture environments as it does physiologically.

### Varying PCL Concentrations to Modulate Oxygen Release Kinetics

In the microparticles, the PCL content was also modified to understand how the hydrophobic barrier controls the oxygen release kinetics of the scaffold. The PCL concentrations ranged from 5% (w/v) PCL to 20% (w/v) PCL, with increasing increments of 1%. In addition, these conditions were also evaluated with respect to the 13.5% (w/v) PCL concentration as used in earlier experiments. The oxygen-releasing content in the microparticles, CaO₂, remained constant at 60 mg/mL in the PCL solution. The mixture stirred for 4 hours for a formation of the PCL-CaO₂ complex. The resulting PCL-CaO₂ complex was utilized as the core of the microparticle. As previously described, our microparticles are fabricated within an aqueous phase consisting of 0.5% (w/v) low molecular weight PVA in deionized water. The oxygen-generating microparticles were assembled using a micropipette technique which involved the dropwise addition of the PCL-CaO₂ solution to the prepared PVA solution under magnetic stirring at 920 rpm. All parameters were optimized to yield an average microparticle size of 100 μm.

### Three-Dimensional (3D) Cell Encapsulation in Oxygen-Generating Scaffolds

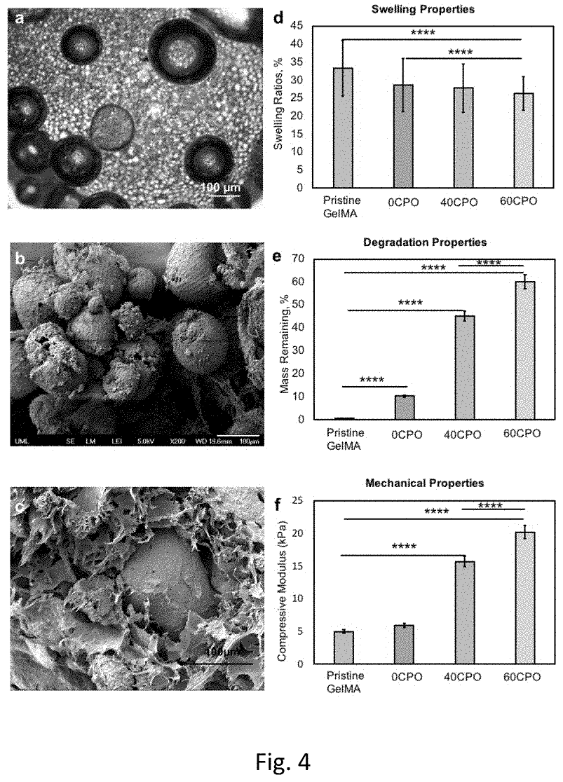

We assessed the cytocompatibility of the oxygen-generating scaffolds with three different cell types. Specifically, cardiac fibroblasts, 3T3 fibroblasts, and L6 rat myoblasts were encapsulated in 3D within the scaffolds. These cell types were encapsulated in separate hydrogel precursor solutions at seeding density of 5×10⁶ cells/mL. The hydrogel precursor solutions were prepared for 5% (w/v) GelMA for each scaffold along with the addition of 0, 40, and 60 mg/mL CaO₂ in PCL. The cells were trypsinized, transferred into a conical tubed, and centrifuged to form a cell pellet. From the pellet, the cell count was obtained to determine the amount cells to resuspend in the prepolymer solutions. The cell-laden prepolymer solution was photocrosslinked at the bottom of a 96-well plate. The 3D-encapsulated scaffolds are fabricated using a UV power of 700 mW/cm² (FIG. 4). These scaffolds were maintained in a prolonged cell culture period of five weeks under induced hypoxia. The various cell types and culture conditions were analyzed for their oxygen-release content, mechanical properties, viability, proliferation, cytotoxicity, and apoptosis.

### Cell Viability and Morphology

The tissue scaffolds were maintained using a cell culture media of DMEM supplemented with 10% (v/v) fetal bovine serum (FBS) and 5% (v/v) penicillin/streptomycin. The cell cultures remained in an incubator at 37° C. with 5% carbon dioxide (CO₂). This cell culture media was changed every 2-3 days. From this in vitro culture, the tissue scaffolds were studied for viability and metabolic activity using a commercial Alamar blue assay. Briefly, the Alamar blue assay reagents incubated with the cells for 4 hours according to the manufacturer's protocol. The colorimetric results were analyzed using a micropipette reader. The fluorescence values were read using 560 nm/590 nm (Ex/Em) setting.

The cytotoxicity results were examined through a commercial lactate dehydrogenase (LDH) cytotoxicity assay. In summary, a 25 μL of supernatant from the cell culture was transferred into a 96-well plate; each sample reacted with the provided reaction analyte in a 1:1 ratio. After 30 minutes, the provided stop solution was added. The absorbance is read using a spectrophotometer set at wavelength of 560 nm. As per previous experiments, four replicates were utilized for each scaffold composition. In addition, we analyzed caspase 3 and 7 activities using a commercial Caspase Glo 3/7 assay by Promega. Similarly, the reaction substrate was added to the samples in a 1:1 ratio and reacted for 45 minutes under dark conditions. The provided stop solution was added after 40 minutes to quench the reaction for luminescence readings.

### Statistical Analysis

GraphPad Prism 6.0 (La Jolla, Calif., USA) was used for all conducted statistical analyses. The results were analyzed by performing a one-way ANOVA. Bonferroni post hoc tests were completed to analyze the statistically significant differences. A p-value <0.05 was considered to be a statistically significant difference in all shown analyses. All values are represented as averages±standard deviation (*p<0.05, **p<0.01, ***p<0.001, and ****p<0.0001).

### Results

The oxygen release kinetics, cellular responses, and biocompatibility of our oxygen-generating scaffolds were evaluated extensively in this work. This section describes the results obtained from the mechanical characterization, the biological performance with diverse cell types, and the oxygen-release profiles of each oxygen-generating scaffold composition.

### Synthesis and Characterization of Oxygen-Generating Scaffolds

The oxygen-generating scaffolds were synthesized by reinforcing a gelatin-based hydrogel with oxygen-releasing microparticles. Using a simple emulsification technique, these particles included varied CaO₂ in PCL concentrations which were then utilized to fabricate distinct scaffold compositions. Based on SEM imaging, it was determined that these microparticle fabrication process yields particles with a mean diameter of 100 μm. The SEM images also revealed the topography and microparticle integration within the GelMA matrix. Other physical characterization also demonstrates modular swelling, degradation, and mechanical behavior based on the scaffold composition. In swelling tests, the physical property decreased accordingly to the increased CaO₂ content in the microparticle. This experiment determined swelling ratios of 33.34%±7.8, 28.58%±7.39, 27.87%±6.7, and 26.31%±4.6 for the Pristine GelMA, 0CPO, 40CPO, and 60CPO scaffolds, respectively. Similarly, the degradation behavior was also concentration dependent. After 12 hours, the percent mass remaining increased in response to the increased CaO₂ content in the microparticle with 0.76%, 10.32%, 45.2%, and 60.1% for the Pristine GelMA, 0CPO, 40CPO, and 60CPO scaffolds, respectively. In mechanical analysis, the increasing oxygen-releasing content in the microparticles has positive effect in improving the compressive modulus of the material. In particular, the compressive moduli were 5.01, 5.9, 15.7, and 20.2 kPa for the Pristine GelMA, 0CPO, 40CPO and 60CPO scaffolds, respectively. Therefore, these physical properties are tunable based on the microparticle composition which in turn affects the ultimate oxygen-generating scaffold composition.

### Cellular Response and Biocompatibility of Oxygen Generating Scaffolds

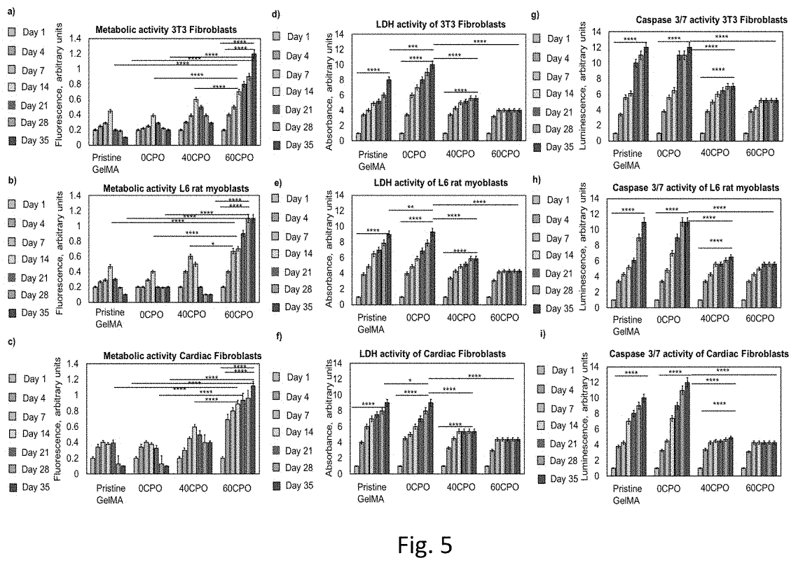

The biocompatibility of the oxygen-generating scaffolds was evaluated in vitro through the Alamar blue assay, LDH assay, and Caspase Glo 3/7 activity assay. Specifically, 3T3 fibroblasts, L6 rat myoblasts, and cardiac fibroblasts were 3D encapsulated within the oxygen-generating scaffolds. FIG. 5 shows the results of these assays for Pristine GelMA, 0CPO, 40CPO, and 60CPO scaffolds.

### Metabolic Activity of Various Cell Types

The metabolic activity across cell types 3D encapsulated in the oxygen-generating scaffolds demonstrated unique behaviors over 35 days in in vitro tissue culture (FIG. 5a-c). The effects of the scaffold compositions on the metabolic activity was assessed at various time points through the Alamar blue assay. These increases in metabolic activity were mainly demonstrated under hypoxic conditions with catalase included in the cell culture medium. For example, the 60CPO group with catalase exhibited the highest increase in metabolic activity of 3T3 fibroblasts at day 14 than other tested scaffold compositions and tissue culture conditions (FIG. 5a). After day 14, the results of this assay show that other scaffolds compositions demonstrated a lower capacity to support the metabolic activity of the 3T3 fibroblasts. In comparison, the 40CPO scaffolds with 3T3 fibroblasts demonstrated increasing metabolic activity up to day 14, and a decline in metabolic activity past this time point. Similarly, the L6 rat myoblasts in scaffolds composed of lower oxygen-generating content exhibited increasing metabolic activity but only up to day 7 in vitro (FIG. 5b). Again, there was particular success in the 60CPO group which showed increased metabolic activity up to day 21. In 60CPO scaffolds, the metabolic activity levels off after day 21, and continues to decrease over time to day 35. Therefore, the metabolic activity of the L6 rat myoblasts in the 60CPO scaffold remained the highest than other oxygen-generating scaffold compositions. On the other hand, primary cardiac fibroblasts showed increasing metabolic activity up to day 14 in Pristine GelMA, 0CPO, and 40CPO with the exception of the 60CPO condition (FIG. 5c). According these results, the 60CPO is most suitable for cell types evaluated here to maintained higher metabolic activity over time than other tested compositions.

### Lactate Dehydrogenase (LDH) Activity for Measuring Cytotoxicity

The cytotoxicity results of this oxygen-generating material were assessed through the LDH activity of the 3D encapsulated cells in cell culture. The results demonstrate that the Pristine GelMA, 0CPO, and 40CPO scaffolds elicited a significant increasing trend of LDH activity in the 3T3 fibroblasts during the 35-day in vitro study (FIG. 5d-f). In contrast, the were modest effects with only steady increase in the LDH levels in 3T3 fibroblasts in the 60CPO composition. This result is also consistent and comparable to trends in the LDH activities of the L6 rat myoblasts and the primary cardiac fibroblasts encapsulated in Pristine GelMA and 0CPO. Interestingly, in the cardiac fibroblasts, there was an observed steady increase in LDH activity in response to the 40CPO scaffolds and no significant increase scaffold in the 60CPO scaffold. Therefore, a desirable lower LDH activity and cytotoxic effects are found in scaffolds with higher oxygen-generating content for primary cardiac fibroblasts.

### Caspase Glo 3/7 Assay for Measuring Apoptosis

Over the 35-day in vitro study, both caspase 3 and 7 activity demonstrated an increasing trend in response to oxygen-generating scaffolds (FIG. 5g-i). Specifically, there was a modest response found in 3T3 fibroblasts 3D-encapsulated in the 40CPO scaffold composition. Contrarily, the Pristine GelMA and 0CPO scaffolds demonstrate lower caspase activity in comparison to 40CPO and 60CPO scaffolds. There was no significant change in the caspase activity found in the study for the 60CPO composition. This cellular behavior is also consistent in scaffolds encapsulating the L6 rat myoblasts and primary cardiac fibroblasts, suggesting that caspase activity may be irrespective to the cell types assessed here.

### Oxygen Release Kinetics of Oxygen-Generating Scaffolds

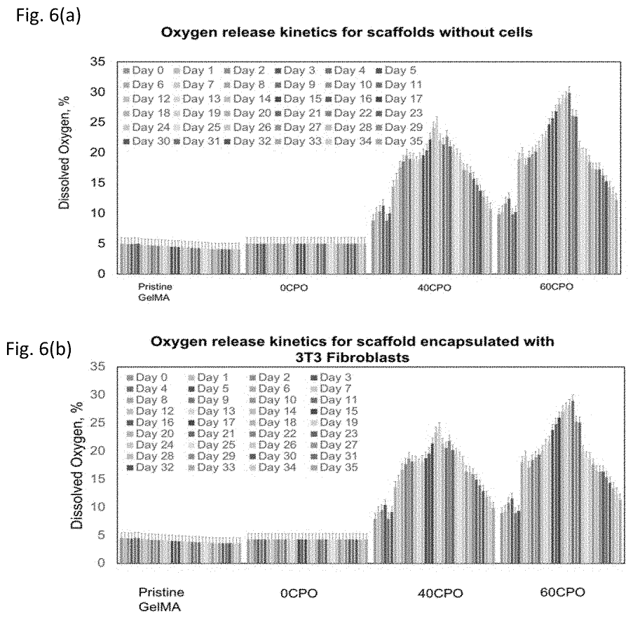

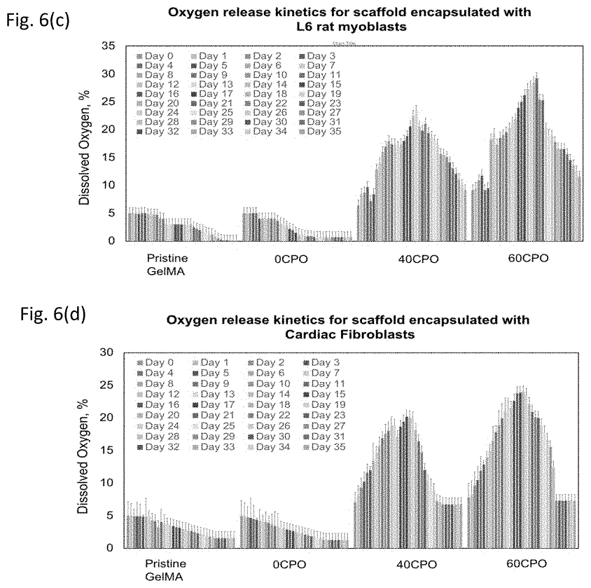

The results oxygen-release kinetics were extrapolated from measurements of oxygen-release in the tissue culture medium. These scaffolds were cultured under hypoxia with the addition of catalase at 1 mg/mL in vitro for 35 days. Furthermore, the tissue culture conditions also varied by cell type encapsulated within scaffold, and includes either 3T3 fibroblasts, L6 rat myoblasts, primary cardiac fibroblasts, or no cells. The peak oxygen release differed between oxygen-generating scaffolds with and without cells, and between different cell types. The scaffolds culture devoid of cells demonstrated a higher average peak oxygen release across all compositions than scaffolds containing 3D-encapsulated cells (FIG. 6(a)-6(d)). Without the presence of cells, the peak oxygen release is shown at later time points in the extended in vitro culture period (FIG. 6a). Specifically, both the Pristine GelMA and 0CPO scaffolds show approximately the same peak oxygen release at day 1 (˜5.02%). In contrast, the 40CPO and 60CPO scaffolds containing cells reached more than a 4-fold increase in peak oxygen release which occurred at later times points in vitro. Specifically, these measurements were 22.71% (day 22) and 29.9% (day 22) in the 40CPO and 60CPO scaffolds, respectively. According to the results, there is concentration dependency between the peak oxygen release and the concentration of CaO₂ in the scaffolds.

In particular, the scaffolds containing microspheres with a lower in CaO₂ content showed a lower capacity to the release more oxygen over time. The day 1 measurements across the various 3T3 fibroblasts tissue constructs and cell culture conditions remained within the range of 4.27%-4.52%. In L6 rat myoblasts and primary cardiac fibroblasts, the initial measurements on day 1 remained at −5.02% oxygen release. For Pristine GelMA and 0CPO scaffolds, the day 1 oxygen release measurements also represent the peak oxygen release which declines over time. In 3T3 fibroblasts, the decline in oxygen release in Pristine GelMA and 0CPO is minimal as it continuously decreases to 3.4%-3.59% by day 35 (FIG. 6(b)). The oxygen release trends between 3T3 fibroblasts in Pristine GelMA and 0CPO are comparable to trends found in these scaffolds without cells. The data also supports that cell type affects the kinetics which is shown in the overall trend in these scaffold compositions with L6 rat myoblasts and primary cardiac fibroblasts (FIG. 6(c)-6(d)). In particular, there is a significant decline from ˜5.02% to ˜0.1-1.5% within these tissue culture systems, suggesting the oxygen consumption rate were higher in the L6 rat myoblasts and primary cardiac fibroblasts than 3T3 fibroblasts. Within 40CPO and 60CPO scaffolds, we anticipate our findings support that the scaffolds are also compatible with other cell types which possess similar oxygen consumption rates (JO₂).

As expected, the 40CPO and 60CPO contained oxygen-releasing microspheres which improves the oxygen kinetics over time. These findings are similar to the kinetics found in these scaffold compositions devoid of cells in FIG. 6(a). In FIG. 6(b), the peak oxygen in release of 3T3 fibroblasts in 40CPO and 60CPO groups were 24.1% by day 19 and 29% by day 21, respectively. Our findings support that oxygen release kinetics are largely dictated by cell type and scaffold composition. Comparably, there is over a 4-fold improvement in the peak oxygen release in primary cardiac fibroblasts with a peak oxygen release of 20.2% (day 17) and 23.9% (day 18), respectively (FIG. 6(d)). This behavior is also seen in the L6 rat myoblasts 3D-encapsulated in these scaffold conditions. The peak oxygen release is presented at 23.3% at day 19 and 29.2% at day 21, in the 40CPO and 60CPO scaffolds (FIG. 6(c)). The findings demonstrate that these oxygen-generating scaffolds can deliver sustained oxygen release under hypoxic conditions. Importantly, the oxygen release kinetics can be controlled based on scaffold composition to adjust to particular cell type. These features of our tissue construct recapitulate desired properties in tunable tissue culture systems for extended periods.

### Effect of Varying PCL Concentration on the Cumulative Oxygen Release

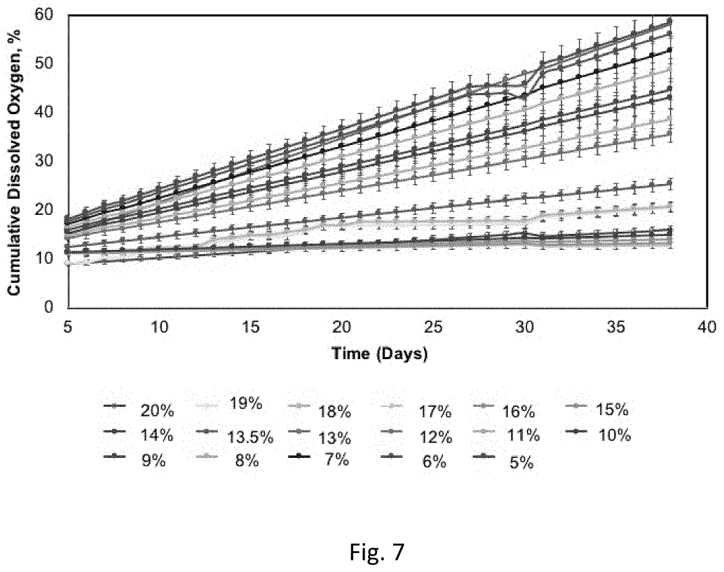

The PCL component of the oxygen-releasing microparticles serves as the hydrophobic barrier to control the hydrolysis reaction that occurs between CaO₂ and the water content in the microenvironment. We varied PCL concentration in the microparticles to understand its effect on the overall oxygen release potential and kinetics in the scaffolds (FIG. 7). Specifically, we used a PCL concentration range of 5% to 20% (w/v) in chloroform, increasing in increments of 1% (w/v). In addition, we also included the previously utilized 13.5% (w/v) PCL in chloroform concentration. To these solutions, the CaO₂ was emulsified at a 60 mg/mL concentration in PCL as previously described. These modified microspheres reinforced the synthesized GelMA precursor solution to fabricate oxygen-generating scaffolds. The results were generated from scaffolds cultured without cells under induced hypoxia and catalase supplemented media. According to the results, the lower PCL concentrations of yielded greater oxygen-release at more rapid rates than at higher PCL concentrations. However, we also attribute the distinct oxygen-release profiles to variety of factors, including the cell type, its oxygen consumption rate, the cell density and the concentration and crosslinking density of the hydrogel polymer. Due to mass transport constraints, we expect that the oxygen release kinetics have an additional dependency on the scaffold's dimension, microparticle and cell distribution, and cell culture media.

### Effect of Oxygen-Generating Scaffolds on pH of Cell Culture Media

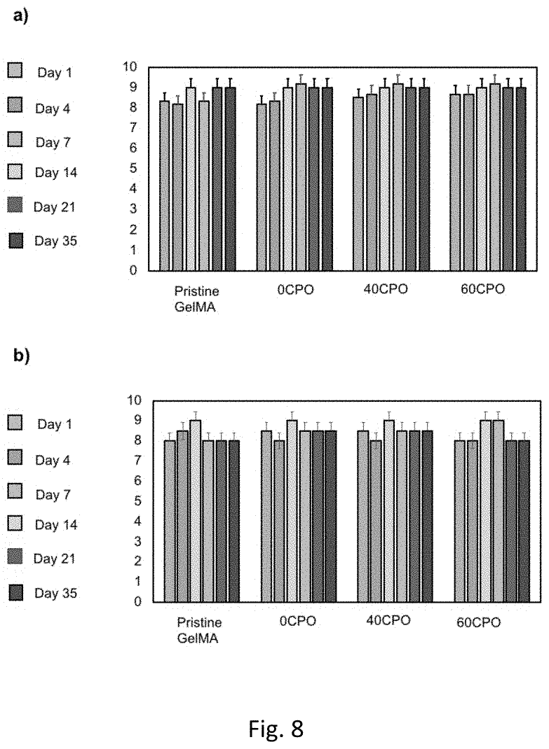

As a critical factor in cellular response and function, we assessed the change in the pH of cellular environment over time with oxygen-generating scaffolds in vitro (FIG. 8). In hydrolysis, one of the byproducts is H₂O₂ which further degrades to form oxygen and water. However, it is also important to consider and characterize the other byproduct, calcium hydroxide (Ca(OH)₂) which can alter the pH of microenvironment. The pH of the supernatant in the cell culture media was measured on days 1, 4, 7, 14, 21 and 35 with scaffolds containing or devoid of primary cardiac fibroblasts to study this effect. Across all cell culture conditions, there were no significant changes in pH in the presence of the oxygen-generating scaffolds with or without cells 3D encapsulated. For Pristine GelMA devoid of cells, the pH remained in the range of pH 8-9 through the 35-day in vitro study. This behavior is also seen in the 0CPO composition. Furthermore, both the 40CPO and 60CPO scaffolds were also similar in that the pH remain between pH 8-8.5 during the experiment. With cells encapsulated, the pH ranges mainly within pH 8 and 8.5 in Pristine GelMA. To note, on day 7, there was a measurable increase to pH 9 before returning to the pH 8-8.5 range. Again, in the 0CPO scaffolds remained in the range pH 8-8.5 with primary cardiac fibroblasts. Similarly, the 40CPO and 60CPO scaffolds remained primarily in the pH 8.5 range with increase to pH 9 between days 4 and 7. Although the changes in pH are measurable, the overall pH remains stable across all scaffold compositions and show no significant increase over the 35-day culture period overall.

### Synthesis, Characterization, and Mechanical Testing of Oxygen-Generating Scaffolds

The synthesized oxygen-releasing microspheres served as the vehicle to deliver optimum partial pressure of oxygen in the microenvironment within tissue scaffolds. The microspheres particle sizes include a diameter distribution range of 50-250 μm. The factors that governed this behavior include the size of these oxygen-releasing microspheres and their (w/v) ratio within the scaffolding biomaterial matrix. Using these parameters, a mean microparticle size of 100 μm was determined as the optimal condition to microencapsulated at a 13.5% (w/v) in the GelMA prepolymer solution. This determined average size and (w/v) ratio was optimized to maintain adequate oxygen availability to cells seeded at a 5×10⁶ cells/mL density. Furthermore, this microsphere composition has a direct effect on the biomaterial properties of the oxygen-releasing scaffold. Therefore, the other physical properties such as swelling, degradation, and compressive strength are also in turn affected by material composition (FIG. 4). Moreover, the techniques involved in the scaffold synthesis yielded unique crosslinking densities, stiffnesses, and porosities. As in other tissue construct types, these material properties largely impact cell viability, proliferation, and cell spreading within the 3D scaffold matrix. At a cellular level, these material characteristics offer biological, chemical, and mechanical cues that will dictate mechano-transduction, guided growth, and differentiation. Therefore, this work robustly covers physical, chemical, and biological analyses to understand the material's behavior.

The results of this work support these important biomaterial properties that facilitate proper tissue construct viability and cellular functions. The swelling analysis demonstrated that the behavior decreases with increasing concentrations of CaO₂ in PCL, and therefore, can be controlled by adjusting this component. As hypothesized, these oxygen-generating scaffolds include hydrogel polymer networks that are hydrophilic which can absorb and retain water. We attribute to the concentration dependency of the swelling behavior to the amount of volume occupied in the hydrogel matrix that is hydrophilic. For instance, in scaffolds that reinforced with microspheres that have higher concentrations of CaO₂ in PCL, there is less hydrophilic content than in scaffolds that are reinforced with particles with lower concentrations of CaO₂ in PCL.

Similarly, the degradation analysis revealed a relationship between percent mass remaining was and the scaffold composition. Specifically, percent mass remaining also increased with increased CaO₂ in PCL. In comparison, oxygen-generating scaffolds reinforced with microspheres containing more CaO₂ content yielded faster degradation rates. It is important to consider that this analysis utilized collagenase type II to facilitate enzymatic degradation of the gelatin component of the scaffold. Therefore, scaffolds with higher concentrations of CaO₂ in PCL possessed a lower amount of degradable gelatin content. Conversely, the scaffolds reinforced with microspheres with lower CaO₂ content included higher amounts of degradable gelatin content. Other findings shown in mechanical testing experiments demonstrated another correlation between the material composition and compressive modulus of each scaffold composition. Our findings support that mechanical strength is another physical characteristic that can also be modified during the microparticle synthesis. Further investigation using SEM imaging revealed the morphology and pore structure of oxygen-generating scaffolds. In particular, there is lace-like surface covering the microparticles throughout the scaffold. Overall, these findings demonstrated how the synthesized oxygen-releasing microspheres interface with the hydrogel matrix and affect the material properties.

### Oxygen Release Kinetics

The oxygen-generating scaffold compositions provided unique oxygen-release profiles. The kinetics found here revealed that the peak release is greater and occurs at later time points in vitro with increased concentration of CaO₂ in the PCL. This behavior is expected based on the dynamics involved in the breakdown of CaO₂ via hydrolysis. With increased CaO₂ content, there is more reactant available to undergo hydrolysis, and will thus lead to more oxygen release over time. The peak oxygen release was determined in hypoxic cell culture conditions with the addition of catalase. After this reactant is depleted, a decline will eventually occur following the peak release. The oxygen-generating scaffolds with and without microencapsulated cells presented this oxygen release behavior. However, the average amount oxygen release at peak measurements was found higher in scaffolds without microencapsulated cells. This finding is expected as there is a lack of cells to consume the dissolved oxygen in the cell culture media during the prolonged in vitro study. Therefore, under the conditions with 3D-encapsulated cells, the lower amount of dissolved oxygen is expected.

These oxygen release kinetic are also controlled by the hydrophobic barrier, PCL, in the microparticles. To understand this aspect, the concentration of PCL in the microspheres was varied while holding CaO₂ in the particles at a constant concentration (i.e. 60 mg/mL). Using increasing range of 5-20%, a gradual and predictable cumulative oxygen release over time is shown in FIG. 7. In particular, there is a positive correlation between the PCL concentration and the oxygen release over time. Consequently, it is possible to both adjust the amount of oxygen-releasing content, CaO₂, as well the hydrophobic barrier, PCL, in the microparticles for fine-tuned scaffold properties.

### Metabolic Activity of Microencapsulated Cells

The biological performance was improved in scaffolds reinforced with microspheres composed of higher CaO₂ content. The results of the Alamar blue assay support that the 60CPO composition provided the most favorable microenvironment for the studied cell types. However, the Pristine GelMA and 0CPO compositions possessed similar results in metabolic activity levels. As these scaffolds are devoid of oxygen-release content, the cell culture systems are deficient of oxygen supply under induced hypoxia. However, there are observable differences between the metabolic activities of the 3T3 fibroblasts, L6 rat myoblasts, and primary cardiac fibroblasts. In 3T3 fibroblasts, the Pristine GelMA and 0CPO scaffolds gradually increased up to day 14. The L6 rat myoblasts and primary cardiac fibroblasts demonstrated similar results in the Pristine GelMA and 0CPO scaffolds. In contrast, the 3T3 fibroblasts in 40CPO and 60CPO scaffolds both demonstrated increase in metabolic activity past 14 days. However, both the myoblasts and cardiac fibroblasts microencapsulated in 40CPO shows increase in metabolic activity up to day 7 before declining and plateauing for the remainder of the culture period. The 60CPO remained as scaffold composition that supported high metabolic activity across cell types continuously up to day 35. Nevertheless, these diverse cell types are expected to demonstrate unique metabolic activity as they are derived from different sources.

### LDH—Cell Cytotoxicity

The presence of LDH found in the cytoplasm of cells is utilized as a biomarker to determine plasma damage and cytotoxicity. Therefore, the LDH levels are indicative of membrane damage which can determine the cytotoxicity of the biomaterial. The LDH activity of the 3T3 fibroblasts, L6 rat myoblasts, primary cardiac fibroblasts presented higher levels in the Pristine GelMA and 0CPO groups, with rapid increases in levels over time (FIG. 5). These results suggest that these scaffold compositions are most cytotoxic among the scaffold compositions tested. This finding also supports that the addition of oxygen-releasing microsphere has a positive effect on improving the cytotoxicity of the tissue scaffold. The fibroblasts and myoblasts yielded significantly lower LDH levels in the 40CPO and 60CPO scaffolds. These cell types in the 40CPO composition showed both a lower and gradual increase in LDH up to 28 days. In comparison, these lower LDH levels were maintained up to 35 days when 3D-encapsulated in 60CPO scaffolds. Moreover, there is significant improvement in scaffolds in the 40CPO and 60CPO scaffolds. For instance, the LDH levels of the primary cardiac fibroblasts plateau in the 40CPO and 60CPO groups at days 14 and day 7, respectively, rather than continuing to increase. Overall, these biological responses suggest that providing an oxygen-releasing component through these microspheres reduces cytotoxic effects of cellular microenvironment. The results also support that 60CPO scaffold composition yielded the most desirable biological performance in vitro in this work. Based on the results, we anticipate seeing different oxygen consumption profiles depending on the cell type, if the studied was modified to test scaffolds with the same cell seeding densities and of identical chemical compositions.

### Caspase Assay for Presence of Apoptosis

The caspase assay revealed the potential for apoptosis in using these oxygen-generating tissue scaffolds (FIG. 5). In this assay, the reaction substrate binds to DEVD which produces a detectable and measurable luminescence that can be correlated to the amount of apoptosis in a particular cell culture. In Pristine GelMA and 0CPO, the 3T3 fibroblast, L6 rat myoblasts, and primary cardiac fibroblasts demonstrated increasing apoptotic activity over time. This finding is expected as this tissue scaffolds are entirely oxygen-deprived under induced hypoxia. Moreover, this analysis is consistent with lower metabolic activity and higher LDH activities found in these compositions. At later time points in in vitro cell culture, such as at day 21, caspase activity dramatically increased. Among the cell types investigated, the L6 rat myoblasts demonstrated to be the most susceptible to apoptosis when microencapsulated in Pristine GelMA and 0CPO under induced hypoxia. Overall, this apoptotic behavior is significantly improved in 40CPO and 60CPO scaffold. Across the cell types, caspase activity decreases over time but eventually increases at day 28. As expected, the 60CPO scaffold supported all cell types in long-term cell culture with preeminently low caspase activity over time. After day 21, caspase activity plateaus in the 60CPO groups, and stops its increasing trend in apoptotic activity. Again, this behavior coincides with previous experiment as LDH levels remained constant after day 14 for this cell culture condition. A distinguishable characteristic is shown in the primary cardiac fibroblasts which maintained constant caspase activity after day 7 in both the 40CPO and 60CPO groups. These findings support that the oxygen-generating scaffolds support cell viability across various cell types under hypoxic conditions.

### Effect of pH

The oxygen-generating scaffolds presented no significant changes in the in vitro cell culture environment. Specifically, the pH of the supernatant of the cell culture remained stable throughout the 35-day tissue culture period. Therefore, the byproducts of hydrolytic degradation, Ca(OH)₂ and H₂O₂, do not modify the pH of the cell culture system detrimentally. These byproducts are essentially temporary reaction intermediates, which may result in a more basic pH of the supernatant. However, as expected, the in vitro cell culture with the pH of Pristine GelMA scaffold which lacked any CaO₂ had no change in pH. Generally, DMEM in the cell culture often contains supplements of with sodium bicarbonate regardless, which results in more basic cellular environments. We have demonstrated the capitalizing of the hydrolytic degradation of CaO₂ does not compromise a stable cellular microenvironment in vitro.

### Conclusion

We developed and robustly characterized novel oxygen-generating scaffolds to provide an essential source of oxygen. Ultimately, this oxygen is utilized for the normal function and regeneration of cells and tissues. Our tissue scaffolds consist of a gelatin-based hydrogel matrix, that is reinforced with oxygen-releasing microparticles. Using an emulsification approach, we have developed a facile and adjustable microparticle synthesis process. Both the oxygen-releasing and hydrophobic constituents can be modified to fine tune a microsphere to different CaO₂ concentrations, with various oxygen-release kinetics, and overall a myriad of oxygen-generating scaffolds. The proposed oxygen generating scaffolds were able to release oxygen consistently for 4 weeks and provided lasting optimal dissolved oxygen levels for up to 5 weeks in culture media when cultured under induced hypoxia. Other material properties such as compressive moduli and swelling and degradation behavior demonstrated modularity with adjustments in the scaffold composition. The findings demonstrate that these oxygen-generating scaffolds can be tailored to variety of tissue engineering applications. We investigated these implications by analyzing the biological performance of these scaffolds with diverse cell types, including 3T3 fibroblasts, L6 rat myoblasts, and primary cardiac fibroblasts. The in vitro results that support that the addition of oxygen-releasing component has functional benefits for these cells in long-term in vitro culture. Specifically, metabolic activity and apoptotic activity are controllable and improved by oxygen-generating scaffolds, with particular success in the 60CPO condition. Under hypoxic conditions, these scaffolds maintain both tissue construct viability and function, as well as a favorable cell culture system. These controlled microenvironments were achieved, without causing adverse or major changes in pH. In the absence of vasculature, these oxygen-generating scaffolds can offer a promising substitute for delivering oxygen supply continuously for 3D and scaled tissue construct viability and functionality. These tunable oxygen-generating scaffolds can serve as excellent biomaterials to improve the in vivo success of implanted tissue constructs. This technology, along with efforts to improve vascularization strategies, can tremendously advance the in vivo clinical performance of tissue-engineered constructs. These oxygen-generating scaffolds are broadly compatible and modifiable for extended tissue engineering research applications.

### Example 2: Cardiac Tissue

### Materials

Polycaprolactone (PCL) pellets were purchased from Fischer Scientific. Calcium peroxide (CaO₂) was purchased from Sigma Aldrich. Porcine Skin Gelatin 100 g was purchased from Sigma Aldrich. Methacrylic anhydride was obtained from Sigma-Aldrich (St. Louis, Mo.). Dulbecco's phosphate buffered saline (DPBS), Dulbecco's Modified Eagle's Medium (DMEM—low glucose), fetal bovine serum (FBS), trypsin-ethylenediaminetetraacetic acid (EDTA) 0.25%, and penicillin/streptomycin (P/S) were purchased from Gibco (Thermo Fisher Scientific, Inc., Waltham, Mass.). Alamar Blue reagent was purchased from Invitrogen (Grand Island, N.Y.). 2-hydroxy-1-[4-(hydroxyethoxy) phenyl]-2-methyl-1propanone (Irgacure 2959) was purchased from BASF Corporation (Florham Park, N.J.). Lactate Dehydrogenase (LDH) activity kit was purchased from Genesee Scientific. Caspase glo 3/7 assay kit was purchased from Promega. NeoFox Oxygen sensing probe was purchased from Ocean Optics Inc. All reagents were used as received without further purification.

### Synthesis of GelMA

The hydrogel precursor comprised of 5% (w/v) porcine skin gelatin derived Gelatin Methacrylate GelMA (GelMA) and 0.5% (w/v) Irgacure 2959 (photoinitiator). To synthesize the GelMA hydrogel precursor, 10 g of porcine skin gelatin was dissolved in 100 mL of DPBS under constant magnetic stirring at 50° C. To this dissolved gelatin solution, 8 mL methacrylic anhydride (MAA) was added dropwise under constant magnetic stirring. The gelatin mixture with MAA was then allowed to react for 4 hours under constant stirring at 200 rpm at 50° C. The mixture was then diluted with 300 mL of DPBS to stop the methacrylation. The mixture was subsequently dialyzed in nitrocellulose membranes submerged in distilled water for one week under constant magnetic stirring (180 rpm) at 40° C. Further, the dialyzed solution was frozen overnight at −80° C. and then lyophilized for one week to obtain the GelMA prepolymer foam. This prepolymer foam obtained during the freeze-drying process was used to make the prepolymer solutions in the photoinitiator solutions. The photoinitiator solutions were prepared by adding 0.5% w/v Irgacure 2959 in 1×DPBS. The prepolymer solutions were finally prepared by adding 5% w/v of the GelMA prepolymer foam and dissolving it in the photoinitiator solution. This prepolymer solution is then used to develop the UV crosslinked oxygen generating scaffolds.

### Synthesis of Oxygen Releasing Microparticle

Oxygen releasing microparticles were fabricated by using calcium peroxide (CaO₂) as the oxygen generating compound which was encapsulated in a hydrophobic phase made of Polycaprolactone (PCL). To fabricate our oxygen releasing microparticles 13.5% w/v PCL was dissolved in chloroform under constant magnetic stirring at room temperature. CaO₂ was added to this PCL in different concentrations 0, 20, 40, 60, 80 and 100 mg/mL respectively which amounted to net 0, 2.7, 5.4, 8.1, 10.8, and 13.5 mg CaO₂ in the GelMA hydrogel matrix upon encapsulation eventually. This solution PCL and CaO₂ was allowed to form a complex by allowing it to magnetically stir for 4 hours. This served as the first viscous phase solution. Then a second aqueous phase solution was prepared by adding 0.5% w/v low molecular weight PVA which was dissolved in DI water at 80° C. under constant magnetic stirring overnight. Thus, a two-phase system was formed wherein the CaO₂ solution in PCL served as the inner viscous phase and the PVA solution served as the aqueous phase. To fabricate the oxygen releasing microparticles, the PCL solution was added dropwise to the PVA solution under constant magnetic stirring.

The particles were transferred to 15 mL falcon tubes and centrifuged at 800 rpm. The supernatant containing excess PVA solution was removed and the particles were washed three times with chloroform to remove residual PVA. The microparticles were then allowed to dry under a vacuum desiccator for 4 hours until all the chloroform evaporated, and no chloroform was left behind.

The particles were then mixed with the GelMA hydrogel prepolymer solution, and homogeneously, which was further added to the cell pellet which was then resuspended. The cell pellet was mixed homogeneously throughout the microparticle dispersed prepolymer. The oxygen releasing microparticles co encapsulated with the cells within the GelMA prepolymer solution was then pipetted between a 150 μm spacer and UV crosslinked to form the cell laden oxygen generating scaffolds. These were then cultured, and their release profiles were recorded, and corresponding cellular response was studied in tandem. To make oxygen releasing microparticles with different release potentials, the concentration of CaO₂ added was varied. Accordingly, 0, 20, 40, 60, 80, and 100 mg/mL CaO₂ was added to the PCL solution to make the oxygen releasing microparticles with different oxygen release potentials and profiles.

### Fabrication of Oxygen Releasing Scaffolds

To fabricate oxygen generating scaffolds, 13.5% w/v oxygen releasing microparticles were homogeneously mixed with GelMA prepolymer. 40 μL of this prepolymer mix was then pipetted at the bottom of a 96 well plate with and without cells, and UV crosslinked to make our oxygen releasing scaffolds. For the purpose of mechanical characterization, scaled up scaffolds were fabricated by adding 13.5% w/v oxygen generating microparticles (OGMPs) to 100 μL of GelMA prepolymer. This was then pipetted in between a 1 mm thick glass spacer and UV crosslinked. The resulting gels were stored in DPBS and used for swelling, degradation, compression tests and Scanning Electron Microscopy (SEM) imaging.

### Swelling and Degradation Analysis

For swelling analysis, the samples were prepared by pipetting 100 μL of GelMA prepolymer solution with 13.5% w/v oxygen releasing microparticles. The solutions were UV using an Omnicure 52000 (EXFO Photonic Solutions Inc., Ontario, Canada). The UV crosslinking time for each gel condition was optimized to 20 sec, 30 sec, 60 sec, 70 sec, 90 sec, and 140 sec respectively for the Pristine GelMA, 0, 20, 40, 60, 80, and 100 mg/mL CaO₂ in PCL gel conditions respectively. The hydrogels were then submerged in 1×DPBS in petri dishes for 48 hours after which they reach equilibrium swelling. Four replicates were performed for each gel composition. Each gel was weighed upon swelling equilibrium, and the excess liquid was removed with a Kimwipe. Subsequently, each hydrogel was weighed in a pre-weighed Eppendorf tube, frozen, and lyophilized for 24 hours. The Eppendorf tubes were weighed again after lyophilization. The dry weights of the hydrogels were recorded after lyophilization. To determine the swelling ratios the wet weight of the hydrogels for each condition was divided by the corresponding dry weight and this ratio was subsequently converted into a percentage value.

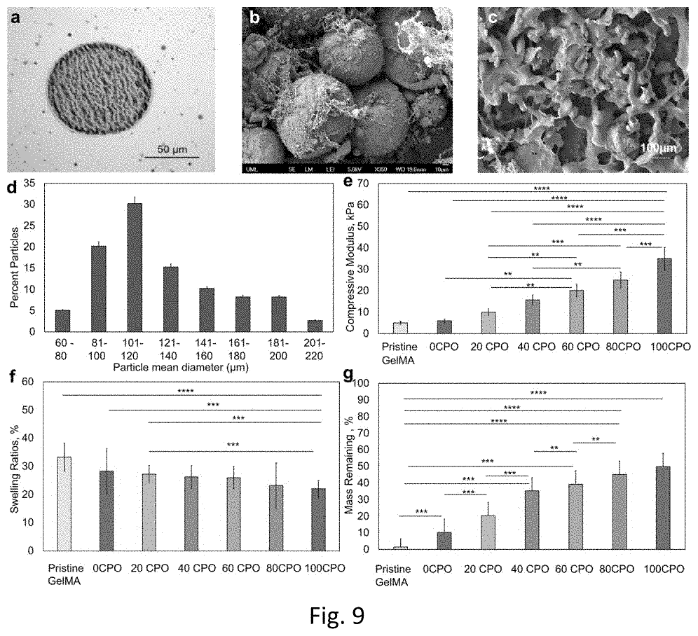

For degradation analysis (FIG. 9g), the hydrogel samples were prepared as previously described. Four replicates were performed for each hydrogel composition. After equilibrium swelling, the gels were transferred into pre-weighed Eppendorf tubes. These samples were then frozen overnight in −80° C. and were lyophilized for 24 hours after which the dry weights were recorded by subtracting the weight of empty Eppendorf tubes from the weight of the lyophilized tubes. After this, 1 mL of PBS was added to the Eppendorf tube to rehydrate the lyophilized gels. After 24 h, the PBS was removed and replaced with 1 mL of 3 U/mL of collagenase type IV in PBS. To initiate the enzymatic degradation, the hydrogel samples were incubated at 37° C. on a shaker at 70 rpm. The mass remaining of each hydrogel was measured at different time points (i.e. 3, 6, 12, 18, 24, 36, and 48 hours). The samples were washed with PBS three times to ensure that the enzyme solution was completely removed at each time point. To obtain the dry weight, the gels were stored at −80° C. overnight before lyophilization. The dry weight of the gel samples was recorded after degradation. The percent mass remaining after degradation was quantified by initial and remaining weights of the hydrogel post the enzymatic degradation. The results were then converted to a percentage value. The end point degradation values were reported as percentage.

### Mechanical Testing

In mechanical analysis, the PCL-CaO₂ microparticle reinforced GelMA hydrogels were prepared using the same process as described. Again, the hydrogels swelled in PBS for 48 h. Prior to the compression test, the samples were shaped using an 8 mm biopsy punch. Any excess or residual liquid on the gels was removed gently by using Kimwipes. The conditions for the compression test included a preload force of 0.0010 N at an isothermal temperature of 23° C., soak time of 1 minute, force ramp rate of 0.1 N/min, and upper force limit was set to 2 N. The compressive modulus of each sample was determined by obtaining the slope in the linear region of the stress-strain curve.

### Porosity

The hydrogel samples were characterized for their pore structure, morphology and size using a scanning electron microscope (SEM)(JEOL 5200 SEM). The SEM image was used for morphological characterization of the PCL-CaO₂ microparticle encapsulated oxygen releasing scaffolds. The gel samples were flash frozen in liquid nitrogen, freeze dried, and gold coated under an argon atmosphere. The SEM images acquired were analyzed using the NIH ImageJ 5.2 a for determining the percent porosity and pore size for gels with 0, 20, 40, 60, 80, and 100 mg/mL CaO₂ in PCL microparticles encapsulated within the GelMA hydrogel prepolymer.

### Oxygen Release Measurements

To study the oxygen release kinetics, all samples were cultured under hypoxia (2% dissolved oxygen) in a StemCell Technologies Hypoxia Chamber. Under hypoxia, the only oxygen source for the cells are the oxygen releasing microparticles co encapsulated within the scaffold matrix. Over the 5-week culture period, the cells consume the oxygen released by the oxygen generating microspheres and therefore we see the dissolved oxygen levels in the with cells, under hypoxia culture group to be slightly lower than the without cells group.

The oxygen levels were then observed in presence and absence of catalase an enzyme known to improve the conversion efficiency of oxygen during the hydrolytic degradation of CaO₂. Catalase is an enzyme that is produced by the liver and it is known to increase the conversion efficiency of hydrogen peroxide (14202), a reaction intermediate during the hydrolytic degradation of CaO₂, to water and oxygen. Next, it was investigated if having catalase in the media would change the release kinetics significantly. 1 mg/mL catalase was added in the culture media and the resulting oxygen release profile was measured.

### Three-Dimensional (3D) Cell Encapsulation in Oxygen Generating Hydrogels

For cytocompatibility studies, H9c2 rat cardiomyocytes were encapsulated in the hydrogel precursor solution at a cell seeding density of 5×10⁶ cells/mL. The hydrogel precursor solutions were prepared using 5% (w/v) GelMA for each hydrogel composition synthesized by the addition of 0, 20, 40, 60, 80, and 100 mg/mL CaO₂ in PCL which amounted to 0, 2.7, 5.4, 8.1, 10.8, and 13.5 mg of CaO₂ in GelMA respectively. To prepare for 3D encapsulation of the H9c2 rat cardiomyocytes, the cells were trypsinised from the flask, transferred into a conical tube, and centrifuged to obtain a pellet. The cell count was obtained from the cell pellet to determine appropriate amounts of cells for homogenous resuspension in the different prepolymer solutions. The cell pellet was resuspended in hydrogel prepolymer solutions which contained the oxygen generating microparticles dispersed homogeneously within. Then, 40 μL of this prepolymer solution containing cells along with the oxygen generating microparticles was pipetted in a 96 well plate and allowed to form a stable disc laden with cells and oxygen generating microspheres within the gel matrix covering the well bottom surface area. The hydrogels were then photocrosslinked using the UV light at 700 mW/cm² power (FIG. 9). The oxygen generating microparticle co-microencapsulated cell laden gels were then cultured under hypoxia for a period of five weeks. The samples were then analyzed for their oxygen content, mechanical properties, viability, proliferation, cytotoxicity, and apoptosis to evaluate their effects on the encapsulated cardiomyocytes in vitro.

### Cell Viability and Morphology

The cells were resuspended in 5% (w/v) GelMA prepolymer added with 0, 20, 40, 60, 80, and 100 mg/mL CaO₂ in PCL at 13.5 w/v concentration in GelMA. The cell-laden GelMA PCL-CaO₂ scaffolds were cultured in a the DMEM medium supplemented with 10% (v/v) fetal bovine serum (FBS) and 5% (v/v) penicillin/streptomycin. The H9c2 rat cardiomyocyte cultures were maintained in a 37° C. incubator with 5% carbon dioxide (CO₂). The media was changed every 2-3 days.

The metabolic activity of the cells was measured by performing an Alamar Blue assay using the standard manufacturer's protocol. The Alamar blue solution, which contained 1 part alarm blue die with 9 parts of DMEM media was incubated with the cells for 4 hours. The colorimetric results were read using a microplate reader in the fluorescence detection mode. The fluorescence values of the resulting supernatant solutions were recorded at 560 nm/590 nm (Ex/Em).

To evaluate the cytotoxic effects of the oxygen-generating scaffolds engineered, the Lactate Dehydrogenase (LDH) cytotoxicity assay was performed according to the standard manufacturer's protocol. 25 μL of sample was pipetted into a 96 well plate and 25 μL of the reaction analyte was added to the solution and allowed to react for 30 minutes. Following this, a stop solution was added to the mixture. Absorbance was subsequently recorded using a spectrophotometer at 490 nm. Four replicates were performed for each scaffold composition.

To check for cellular apoptosis, Caspase Glo 3/7 assay was performed using a Caspase Glo 3/7 apoptosis kit (Promega). The reaction substrate was added to the samples in a 1:1 ratio and allowed to react for 45 minutes protected from light. After 40 minutes, a stop solution was added, and the luminescence of the resulting reacted analyte was recorded which was indicative of any possible change cellular apoptosis over the entire duration of the culture period.

To test the effect of addition of CaO₂ in the scaffolds on the pH of the media, pH strips were used. 20 μL of the supernatant culture media was tested on pH strips to record the pH. The pH measurements were recorded by recoding the color change of the pH strips upon contact with the media that was used to feed the cells encapsulated in different scaffold compositions.

### Statistical Analysis

All statistical analyses were performed using GraphPad Prism 6.0 (La Jolla, Calif., USA). The results were evaluated by performing a one-way ANOVA. The statistically significant differences were analyzed by performing Bonferroni post hoc tests. In all analyses shown, p value <0.05 was considered to be a statistically significant difference. All values are represented as averages±standard deviation (*p<0.05, **p<0.01, ***p<0.001, and ****p<0.0001).

Results

The oxygen generating scaffolds were developed by adding CaO₂ as an oxygen source, at 0, 20, 40, 60, 80 and 100 mg/mL concentrations in a PCL solution. The PCL acts as a hydrophobic barrier which controls the rate at which the water from the surrounding hydrogel matrix reacts with the encapsulated CaO₂, which in turn allows us to control the rate of hydrolytic degradation of CaO₂ and consequently the oxygen release kinetics of our engineered scaffolds. This section summarizes all the analyses performed to characterize the physical and biological properties of these oxygen generating scaffolds and their effects of encapsulated H9c2 cardiomyocytes.

### Synthesis and Characterization of Physical Properties of Oxygen Generating Scaffolds

Oxygen generating microparticles were synthesized using the protocol mentioned in the previous sections with different concentrations of CaO₂ as described in Table 1. The protocol was optimized to yield composite microparticles with an average diameter of 100 μm. FIG. 9 shows the phase contrast image (FIG. 9a), SEM image (FIG. 9b) and the scaffold cross section (FIG. 9c). FIG. 9d shows the size distribution of the oxygen generating microparticles obtained per batch of microparticles synthesized. The protocols were optimized to ensure a maximum batch yield of 30% per batch of microparticles synthesized were 100 μm which were then chosen and isolated and used as the average microparticle size for standardization of all experiments. The stir speeds and batch volumes used were optimized so that most particles in that batch would have an average size of 100 μm. For the cardiac cell experiments, an average particle size of 100 μm was optimized. However, it is possible to optimize the magnetic stirrer speed to yield microparticles between 50-250 depending upon the scalability of the desired application. FIG. 9a, shows the phase contrast image of an oxygen releasing microparticle obtained using an inverted Zeiss microscope. The image shows the particle shape and structure after it was washed with chloroform and cured by vacuum drying. To enable this, the oxygen releasing microparticles were weighed out in Eppendorf tubes at 13.5% w/v concentration with respect to the GelMA prepolymer and the GelMA prepolymer solution was added to the Eppendorf and the particles were resuspended to ensure homogeneous mixing. The oxygen-releasing microparticles were then 3D microencapsulated by pipetting 100 μL GelMA prepolymer solution containing 135 μL of the PCL-CaO₂ microparticle pellet dispersed, between a 150 μm thick spacer mounted on a petri dish. A glass slide was placed over the pipetted prepolymer and then UV crosslinked using an OMNICURE UV lamp. This yielded the 3D encapsulated oxygen releasing hydrogel scaffolds which are shown in FIG. 9. The mean diameters of multiple such oxygen releasing microparticles were measured using Image J 1.52 a to yield a particle size distribution curve as shown in FIG. 9d. This particle size distribution was observed for one batch of 1000 μL of the PCL-CaO₂ solution.

These microparticles were microencapsulated at a 13.5% w/v concentration in a GelMA hydrogel prepolymer to fabricate the oxygen generating scaffolds. To further characterize the physical appearance and mechanical properties of the oxygen releasing microparticles, SEM imaging was performed on the particles using a Field-Emission Scanning Electron Microscope (JEOL JSM 7401F, Peabody, Mass.).

The SEM images of the particles reveal the surface structure as shown in FIGS. 9b and 9c. The mechanical properties of the oxygen releasing microparticles were characterized using DMA compression test. Our results indicate that the compressive modulus of the scaffolds with oxygen generating microparticles encapsulated within the GelMA matrix increases as the concentration of CaO₂ encapsulated in the PCL microparticles increases. The compressive modulus 5.01 kPa, 5.9 kPa, 10 kPa, 15.7 kPa, 20.2 kPa, 25 kPa, 35 kPa was recorded for the Pristine GelMA, 0CPO, 20CPO, 40CPO, 60CPO, 80CPO, and 100CPO groups respectively (Table 1).

We also characterized the swelling and degradation properties of the oxygen generating scaffolds. The swelling ratios of the scaffolds decreased with an increase in concentration of CaO₂ in PCL. The swelling ratios decreased form 33.34%, 28.58%, 28.13%, 27.87%, 26.31%, 22.17%, and 21.83% for Pristine GelMA, 0CPO, 20CPO, 40CPO, 60CPO, 80CPO, and 100CPO groups respectively. The degradation results showed that the scaffolds with highest CaO₂ in PCL had the highest mass remaining at the end point. The results show that by the end point of the degradation experiment, 0%, 10.21%, 20.29%, 35.32%, 38.23%, 43.99%, and 49.22% mass remained for the Pristine GelMA, 0CPO, 20CPO, 40CPO, 60CPO, 80CPO, and 100CPO groups respectively.

### Characterization of Cellular Response

To test the cellular response of the oxygen releasing microparticles the H9c2 cardiac myocytes were microencapsulated within GelMA hydrogel prepolymer along with 13.5% (w/v) PCL-CaO₂ microparticle concentration. The cells were co-encapsulated at a cell seeding density of 5×10⁶ cells/mL along with 13.5% w/v microparticle concentration. Then, 40 μL of gel prepolymer along with cells and the oxygen releasing microparticles was pipetted into 96 well plates and exposed to UV light to be crosslinked. These formed the proposed oxygen releasing scaffolds. The oxygen release over time was measured daily using the NeoFox oxygen sensing probe, which detected the change in the partial pressure of the oxygen dissolved in the supernatant media.

To study the oxygen release kinetics, all samples were cultured under hypoxia in a StemCell Technologies Hypoxia Chamber. Under hypoxia, the only oxygen source for the cells are the co-encapsulated oxygen generating microparticles. We tested the samples with Pristine GelMA 0CPO, 20 CPO, 40CPO, 60CPO, 80CPO, and 100CPO compositions. A cell seeding density of 5 million cells/mL was used to keep the experiments standard across all scaffold compositions.

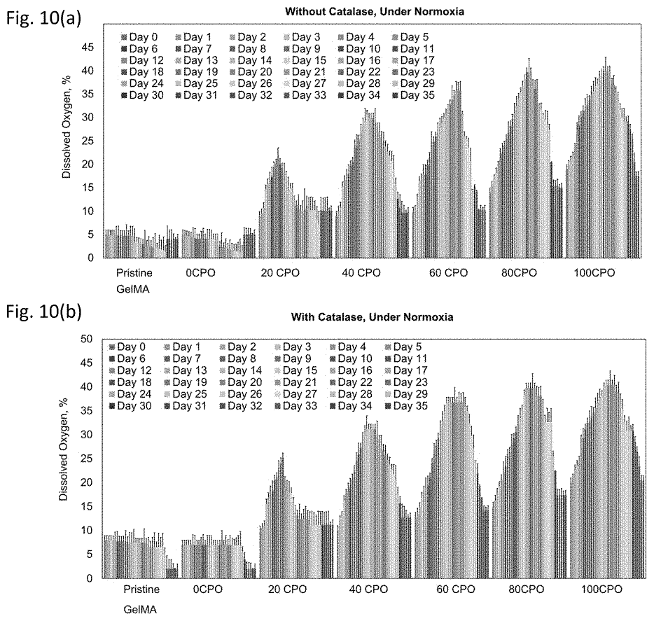

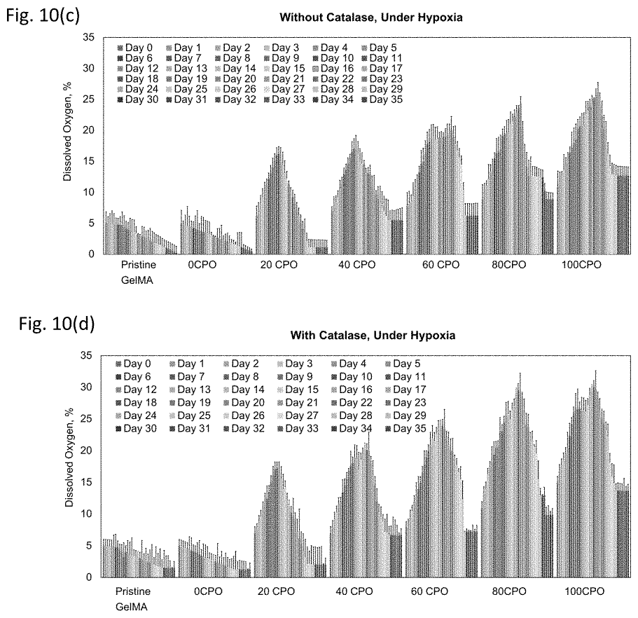

The experiment was performed under normoxia and under hypoxia and also both with and without catalase in media over the 35 day culture period. Over the 35 days, the cells consume the oxygen released by the oxygen generating microparticles, and therefore we see the dissolved oxygen levels in the with cells, under hypoxia group are slightly lower than the without cells group. The dissolved oxygen increased for all scaffold compositions steadily, reached a peak at different time points as indicated in FIG. 10(a)-10(d), and tailed off and lowered.

For the groups Pristine GelMA, 0CPO, 20CPO, 40CPO, 60CPO, 80CPO, and 100CPO a peak dissolved oxygen release of 4.99% from Day 1 through Day 14, 4.89%, 22.07% on Day 9, 29.9% on Day 16, 35.73% on Day 21, 36.17% on Day 21, 39.01 on Day 21 was observed respectively under normoxia without catalase in media. The presence of catalase under normoxia increased the peak dissolved oxygen to 8.023% on Day 0 through Day 14, 7.019% Day 0 through 14, 25.23% on Day 11, 31.25% on Day 14, 36.85% on Day 21, 39.8% on Day 21 and 40.43% on Day 21 for the Pristine GelMA, 0CPO, 20CPO, 40CPO, 60CPO, 80CPO, and 100CPO groups respectively.

To study the isolated effects of the oxygen generating microparticles on the encapsulated cells, the scaffolds were cultured under hypoxia both with and without catalase. The scaffolds cultured Under hypoxia without catalase showed peak dissolved oxygen of 5.023% on Day 0 decreasing through to 0.258% on Day 35, 5.019% on Day 0 decreasing through to 0.504% on Day 35, 16.23% on Day 11, 17.21 on Day 12, 20.1 on Day 22, 24.26% on Day 19, and 26.23% on Day 20 for the Pristine GelMA, 0CPO, 20CPO, 40CPO, 60CPO, 80CPO, and 100CPO groups respectively.

The scaffolds cultured under hypoxia with catalase 5.023% on Day 0 decreasing through to 1.51% on Day 35, 5.019% on Day 0 decreasing through to 1.27% on Day 35, 14.25% on Day 15, 20.21% on Day 17, 23.85% on Day 17, 29.6% on Day 18, and 20.03% on Day 18 for the Pristine GelMA, 0CPO, 20CPO, 40CPO, 60CPO, 80CPO, and 100CPO groups respectively.

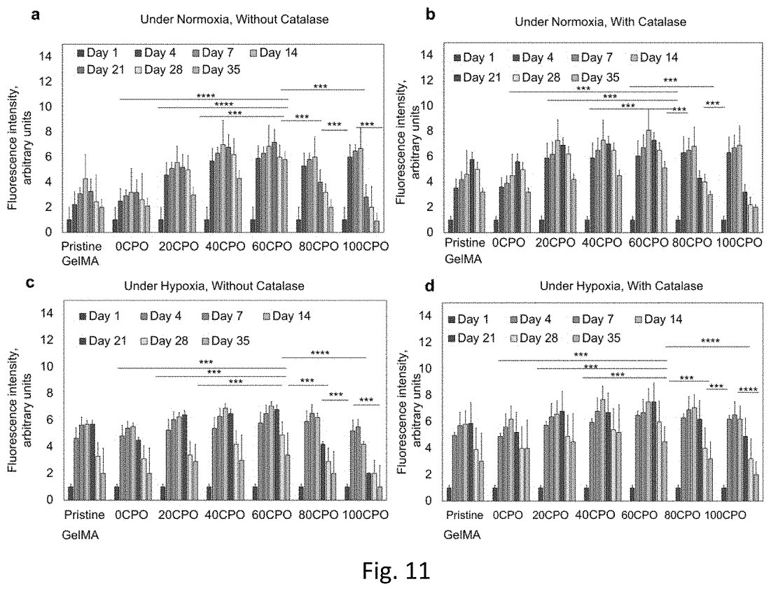

The effects of the oxygen generating scaffolds on the metabolic activity of the H9c2 cardiomyocytes encapsulated were evaluated using the Alamar Blue assay (FIG. 11). The oxygen generating scaffolds with H9c2 cardiomyocytes microencapsulated were cultured under normoxia without and with catalase in media (FIGS. 11a and 11b) and under hypoxia with and without catalase in media (FIGS. 11c and 11d). The metabolic activity was evaluated using the Alamar Blue assay by measuring the fluorescence intensity and the results show that across all groups, the 60CPO group showed the highest metabolic activity.

For the scaffolds cultured under normoxia without catalase (FIG. 11a), the metabolic activity increased across all groups up to Day 14 at different intensities, and subsequently decreased. As expected, 20CPO, 40CPO and 60CPO groups showed higher metabolic activity as compared to the negative control groups of pristine GelMA and 0CPO groups. The 80CPO and 100CPO groups showed a significant decrease in metabolic activity of the cells indicating that excess oxygen damaged the cells.

For the scaffolds cultured under normoxia, with catalase (FIG. 11b), the metabolic activity observed was higher across all groups. The metabolic activity showed a similar trend increase up to Day 14 ad subsequent decrease. 20CPO, 40PO and 60CPO scaffolds showed higher metabolic activity at the corresponding time points as compared to the Pristine GelMA and 0CPO groups, The 60CPO showed the highest end point metabolic activity. The 80CPO and 100CPO scaffolds show a steady decline in metabolic activity after Day 14 indicative of oxidative damage due to excess oxygen.

For the scaffolds cultured under hypoxia, without catalase, (FIG. 11c) the 60CPO group showed the highest metabolic activity across all time points. The 80 and 100CPO scaffolds showed a decrease in metabolic activity, the 20, 40, and 60CPO scaffolds showed higher metabolic activity as compared to the Pristine GelMA and 0CPO groups.

For the scaffolds cultured under hypoxia with catalase (FIG. 11d), the highest metabolic activity was observed across all scaffold compositions for the 60CPO condition. The 20CPO, 40CPO, and 60CPO scaffolds showed higher metabolic activity than the Pristine GelMA and 0CPO conditions. As expected even under hypoxia, the 80CPO and 100CPO scaffolds caused decrease in cell metabolic activity indicating that there is an optimum range of dissolved oxygen which supports cell metabolic activity.

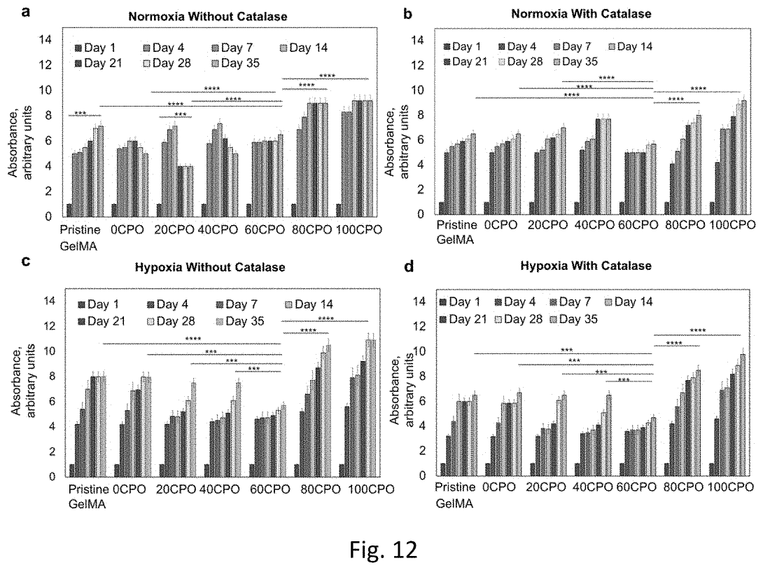

To study the cellular response to the encapsulated H9c2 cardiomyocytes to the oxygen generating scaffolds, an LDH cytotoxicity assay was performed (FIG. 12). The results show an increase in LDH activity for the 80CPO and 100 CPO scaffolds. The 60CPO scaffolds show the least LDH activity thus supporting that 60CPO may be the ideal scaffold composition for this experiment.

For scaffolds cultured under normoxia, without catalase (FIG. 12a), the results show that the LDH activity showed a sharp increase from Day 1 through Day 35 for the pristine GelMA, 0CPO, 80CPO and 100C. The LDH levels remained constant for the 60CPO scaffolds indicating minimum cytotoxicity in this group. The increase in LDH activity in the Pristine GelMA, 0CPO, 20CPO and 40 CPO scaffolds indicate increase in cytotoxicity in absence of sufficient oxygen and for the 80CPO and 100CPO groups the cytotoxicity increased due to excess oxygen which also damages cells.

For scaffolds cultured under normoxia with catalase (FIG. 12b) more cytotoxicity was observed compared to the without catalase group which may be attributed to the excess dissolved oxygen present in the culture media. The 60CPO scaffolds show the least increase in LDH activity. The Pristine GelMA, 0CPO, 20CPO, 40CPO groups show a steady increase in LDH activity over the time points. The 80CPO and 100CPO groups show a sharp increase in LDH activity which indicates damage due to excessive oxygen.

For scaffolds cultured under hypoxia without catalase (FIG. 12c), the 60CPO group showed the least increase in LDH activity which remained fairly constant across all time points. The LDH activity show a significant increase for the Pristine GelMA, 0CPO, 20CPO and 40CPO scaffolds due to lack of sufficient oxygen. The 80CPO and 100CPO conditions showed a sharp increase in LDH activity indicative of oxidative damage.

For the scaffolds cultured under hypoxia with catalase in media (FIG. 12d), the LDH activity showed the least increase in LDH activity across all groups. The 60CPO group showed no significant increase in LDH activity. The Pristine GelMA, 0CPO, 20CPO and 40 CPO showed a steady increase in LDH activity by Day 35. The 80CPO and 100CPO showed a sharp significant increase in LDH activity from Day 1 through Day 35.

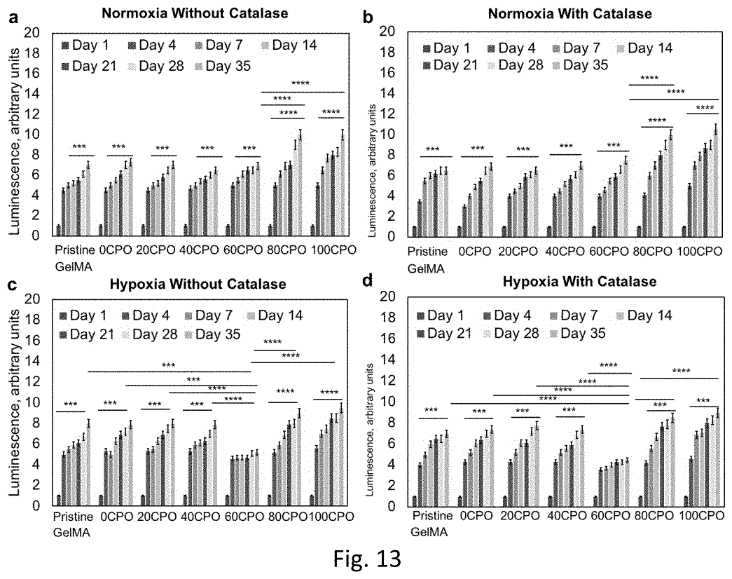

The cellular response of the encapsulated H9c2 cardiomyocytes was further evaluated by checking for cellular apoptosis using the Caspase Glo 3/7 assay (FIG. 13). The reaction produced luminesce which was proportional to cellular apoptosis.

The results show that for scaffolds cultured under normoxia without catalase (FIG. 13a), the Pristine GelMA, 0CPO, 20CPO, 40CPO and 60CPO group showed a slight increase in apoptosis from Day 1 through Day 35. This was however very low compared to the apoptosis observed in the 80CPO and 100CPO scaffolds. This can be attributed to the presence of excessive dissolved oxygen in media causing oxidative damage to the cardiomyocytes.

The results for the scaffolds cultured under normoxia with catalase (FIG. 13b), show a slight increase in apoptosis for the Pristine GelMA, 0CPO, 20CPO, 40CPO and 60CPO groups from Day 1 through Day 35. This was however significantly low compared to the apoptosis observed for the 80CPO and 100CPO groups.

The scaffolds cultured under hypoxia without catalase (FIG. 13c) show a significant increase in apoptosis from Day 1 through Day 35 for the groups Pristine GelMA, 0CPO, 20CPO, and 40CPO due to the lack of sufficient oxygen under hypoxia. This increase was however much lower than the sharp increase in apoptosis observed for the 80CPO and 100CPO groups which cause apoptosis due to oxidative damage to the cells.

The scaffolds cultured under hypoxia with catalase (FIG. 13d), show improved response due to the presence of catalase. The 60CPO group showed the least increase in apoptosis from Day 1 through Day 35. The Pristine GelMA, 0CPO, 20CPO, 40CPO scaffolds show significant increase in apoptosis from Day 1 to Day 35 but is significantly lower compared to the 80CPO and 10CPO groups which show the highest apoptosis across all time points.

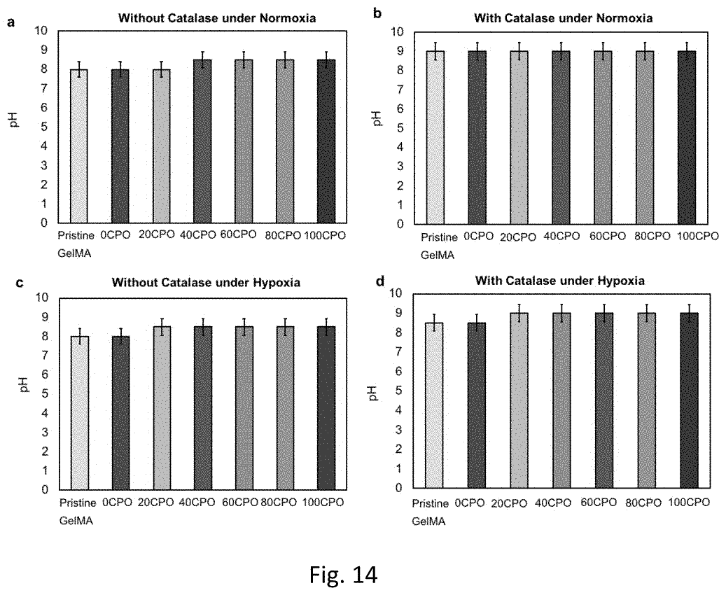

The pH of the media was monitored across all groups (FIG. 14). Our results show that the pH did not increase significantly for scaffolds cultured under all four conditions. As shown in FIG. 14a, the scaffolds cultured under normoxia without catalase, pH of 8, 8, 8, 8.5, 8.5, 8.5, 8.5 was observed for the Pristine GelMA, 0CPO, 20CPO, 40CPO, 60CPO, 80CPO, and 100CPO groups respectively.

For the scaffolds cultured under normoxia with catalase (FIG. 14b) a pH of 9 was observed across all groups Pristine GelMA, 0CPO, 20CPO, 40CPO, 60CPO, 80CPO, and 100CPO groups respectively.

The scaffolds cultured under hypoxia without catalase (FIG. 14c) showed a pH of 8, 8, 8.5, 8.5, 8.5, 8.5, 8.5 for the Pristine GelMA, 0CPO, 20CPO, 40CPO, 60CPO, 80CPO, and 100CPO groups respectively.

The scaffolds cultured under hypoxia with catalase (FIG. 14d) showed a pH of 8.5, 8.5, 9, 9, 9, 9, 9 for the Pristine GelMA, 0CPO, 20CPO, 40CPO, 60CPO, 80CPO, and 100CPO groups respectively.

Collectively the results show no significant change in the pH of the supernatant media because of the presence of the oxygen generating microspheres over the 35-day culture period.

### Discussion

The oxygen generating scaffolds were fabricated and their physical and mechanical properties were characterized. The results indicate that the mechanical properties such as the compressive modulus, swelling and degradation can be controlled by controlling the amount of CaO₂ in PCL. This allows development of highly tunable scaffolds for scalable and highly optimized applications in tissue engineering.

The scaffolds were cultured with H9c2 cardiomyocytes microencapsulated at a cell density of 5 million cells/mL. It was investigated if the presence of catalase in the media would change the release kinetics significantly. The oxygen levels were then observed in presence of 1 mg/mL catalase in media. Catalase is an enzyme that is produced by the liver and it is known to increase the conversion efficiency of hydrogen peroxide to water and oxygen. We therefore expected that it could affect the release kinetics and help achieve a higher release of oxygen.

Our results indicate that presence of 1 mg/mL catalase in media increases the conversion efficiency of the hydrogen peroxide to oxygen and water and therefore we see higher oxygen levels of oxygen as compared to the no catalase in media experiments. The with catalase, with cells groups had lower dissolved oxygen levels as expected, as the cells consume the released oxygen overtime.

It was observed that as the concentration of CaO₂ in the oxygen generating microparticles increased, the corresponding peak dissolved oxygen in media increased. Also, as the CaO₂ concentration increased, the peak oxygen release was observed at later time points, indicative of longer release potentials and slower release rates. The dissolved oxygen was measured for media used to culture the oxygen generating scaffolds under normoxia, with and without catalase, and under hypoxia with and without catalase. The comparison showed us how tunable oxygen release can be achieved using the method of fabrication of oxygen generating scaffolds used here.

The cellular response to the oxygen generating scaffolds was evaluated using the Alamar blue, LDH and Caspase glo 3/7 assays. Based on our results it was evident that a the 60CPO scaffolds proved most favorable for the encapsulated H9c2 cardiomyocytes under all culture conditions. The results show how a threshold of dissolved oxygen in media ensures optimum cell proliferation and metabolic activity. The optimum scaffold response was evaluated in the hypoxia with catalase condition which confirmed that the 60CPO scaffold concentration showed the best results in vitro for the culture conditions used. The presence of catalase in media shows improved cell response in vitro.

The pH of the media was measured across all culture conditions and did not show any significant increase over the 35 day culture period. This indicates and shows the in vitro biocompatibility of the engineered oxygen generating scaffolds.

### Conclusion

In conclusion, the results support the hypothesis that the PCL-CaO₂ oxygen generating microparticles, used with GelMA hydrogels help improve cell proliferation, and metabolic activity under hypoxic conditions. These scaffolds also exhibit good biodegradability and biocompatibility in vitro. The oxygen generating scaffolds were able to provide oxygen in a controlled sustainable manner for up to 4 weeks while providing lasting optimum dissolved oxygen levels for up to 5 weeks under hypoxia and therefore support cell metabolic activity. Overall the results revealed that the 60CPO scaffolds provided the most favorable cellular response when cultured under hypoxia and in presence of catalase in media. Study of the oxygen release kinetics for different scaffold compositions reveal that the 60CPO scaffolds provide the most favorable release profile for the H9c2 cardiomyocytes over the 35-day culture period. The 80CPO and 100CPO scaffolds, on the other hand provided highest dissolved oxygen to the encapsulated cells which in fact proved to be detrimental to normal cell function and increased cytotoxicity and apoptosis. This finding indicates that there is a threshold range of dissolved oxygen required for cells to function in a healthy optimal manner. We would expect this optimal range to vary depending upon the cell type used, their oxygen consumption rate, the cell seeding density, volume of the scaffold all of which are scalable depending on the type of tissue engineering application. Additionally, by controlling the amount of CaO₂ in the oxygen generating microparticles, the concentration of PCL, the w/v ratio of CaO₂-PCL microspheres within the hydrogel matrix, and the seeding density of the cells, it is possible to achieve a highly tunable oxygen release kinetics. These oxygen generating scaffolds prevent burst release of oxygen and help cells overcome hypoxia induced necrosis. These scaffolds allow integration with the cellular microenvironment. The extracellular pH is not significantly impacted as a result of the Ca(OH)₂ precipitated as a reaction intermediate. Further experiments will make efforts to test the impact of these oxygen releasing scaffolds on cell function by studying the expression of cardiac biomarkers that can help further investigation of the biological benefits of using these oxygen releasing scaffolds. With the successful demonstration of biocompatibility and biodegradability of these oxygen generating scaffolds in vitro for H9c2 cardiomyocytes, an in vitro model has been established which can serve as an excellent platform to test its applicability with numerous cell types which make up high oxygen demand tissues. Using this approach could pave way for successful in vivo translation of cardiac tissue constructs. The oxygen releasing biomaterials developed in this work can therefore be an impactful biomaterial for many highly metabolically active and high oxygen demand tissues.

### Example 3: Bone Regeneration

### Materials

Polycaprolactone (PCL) was obtained from Fischer Scientific. Calcium peroxide (CaO₂) was supplied by Sigma Aldrich. Porcine skin gelatin 100 g was acquired from Sigma Aldrich. Methacrylic anhydride (MAA) was obtained from Sigma-Aldrich (St. Louis, Mo.). Dulbecco's phosphate buffered saline (DPBS), Dulbecco's Modified Eagle's Medium (DMEM—low glucose), fetal bovine serum (FBS), trypsin-ethylenediaminetetraacetic acid (EDTA) 0.25%, and penicillin/streptomycin (P/S) were purchased from Gibco (Thermo Fisher Scientific, Inc., Waltham, Mass.). Alamar Blue reagent was obtained from Invitrogen (Grand Island, N.Y.). 2-hydroxy-1-[4-(hydroxyethoxy) phenyl]-2-methyl-1propanone (Irgacure 2959) was acquired from BASF Corporation (Florham Park, N.J.). Lactate Dehydrogenase (LDH) activity kit was purchased from Genesee Scientific. Caspase glo 3/7 assay kit was procured from Promega. NeoFox Oxygen sensing probe was purchased from Ocean Optics Inc. All reagents were used as received without further purification.

### Synthesis of Gelatin Methacrylate (GelMA)

The precursor hydrogel solution composed of 5% (w/v) porcine GelMA and 0.5% (w/v) Irgacure 2959 photoinitiator. The GelMA was synthesized by dissolving 10 g of porcine skin gelatin in 100 mL DPBS under constant stirring conditions at 50° C. Then, 8 mL of methacrylic anhydride (MAA) was added dropwise to this mixture. The gelatin solution added with the MAA were allowed to react for 4 hours under constant stirring at 200 rpm and 50° C. To stop the methacrylation reaction, 300 mL of DPBS was added to dilute the mixture. Subsequently, this mixture was transferred into nitrocellulose dialysis membranes and submerged in distilled water for one week to dialyze under constant magnetic stirring (180 rpm) at 40° C. The dialyzed solution was stored at −80° C. in 50 mL falcon tubes and allowed to freeze overnight. The GelMA foam was obtained by lyophilizing the dialyzed solution for one week. The prepolymer solutions were prepared using this obtained freeze-dried GelMA foam.

### Oxygen-Generating Microparticles Synthesis

The oxygen-releasing microparticles were fabricated by encapsulating calcium peroxide(CaO₂) within PCL as the hydrophobic barrier. The hydrophobic phase was prepared using 13.5% (w/v) PCL dissolved in chloroform under constant magnetic stirring at ambient room temperature. The CaO₂ was added into the PCL at 0, 30, 60, and 90 mg/mL concentrations for obtaining varying oxygen-release kinetics. The solutions continued mixing for 4 hours to allow for the formation of a CaO₂ and PCL complex. The aqueous phase was prepared by dissolving 0.5% (w/v) low molecular weight PVA in deionized water under constant stirring at 80° C. The viscous PCL-CaO₂ solution was added dropwise to the PVA solution under constant magnetic stirring at 920 rpm. This technique yielded the oxygen-releasing microparticles. For each experiment, ten batches of the oxygen-releasing particles were performed. To separate the phases, the oxygen-releasing microparticles along with its surrounding medium were transferred into a conical tube and centrifuged at 800 rpm. The microparticles were washed three times with chloroform to remove residual PVA, and then, the supernatant containing excess PVA solution was removed. The microparticles were placed under a vacuum desiccator for 4 hours to dry and evaporate the remaining chloroform for use.

### Oxygen-Generating Scaffold Fabrication

The synthesized microparticles were used to reinforce GelMA for producing the oxygen-generating scaffolds. These scaffolds were fabricated by homogenously mixing 13.5% (w/v) oxygen-releasing microparticles with the 5% (w/v) GelMA prepolymer. From this prepolymer mixture, 40 μL was pipetted to the wells of a 96-well plate. The polymer precursor solutions were photocrosslinked using ultraviolet (UV) light at 700 mW/cm² (Omnicure S2000, EXFO Photonic Solutions Inc., Ontario, Canada). The oxygen-generating scaffolds were also scaled to 100 μL volume for physical characterization. For these samples, the solution was pipetted in between 1 mm thick glass spacer for UV-crosslinking. The resulting gels were stored in PBS and used for swelling, degradation, compression tests, and scanning electron microscopy (SEM) imaging.

### Swelling and Degradation Analysis

As described in the previous section, the GelMA prepolymer solution with 13.5% (w/v) oxygen releasing microparticles were utilized in swelling analysis. The optimized UV crosslinking times for 0, 30, 60, and 90 mg/mL gel conditions are respectively: 20, 40, 80, and 130 seconds. The hydrogels were submerged in DPBS in petri dishes for 48 hours to reach equilibrium swelling. Four replicates were completed for each gel composition. Each hydrogel was removed from the DPBS and excess liquid were carefully blotted off using a Kimwipe. The hydrogels were then transferred to Eppendorf tubes, weighed, frozen overnight, and lyophilized for 24 hours. Following lyophilization, the Eppendorf tubes were weighed again. The wet weights of the gels were divided by their corresponding dry weights to determine the swelling ratios. These ratios were then reported after converting into percentage values.

In the degradation analysis, the hydrogel samples were prepared as described in the previous sections for all hydrogel compositions. The hydrogels were allowed to swell overnight before being transferred into pre-weighed Eppendorf tubes. The samples were stored overnight at −80° C. and then lyophilized for 24 h. The dry weight of the sample was determined by subtracting the weight of the empty Eppendorf tube from the combined weight of the sample and Eppendorf tube. The lyophilized gels were then rehydrated using 1 mL of PBS. After 24 hours, the PBS was removed and replaced with 1 mL 2.5 U/mL of collagenase type II in PBS. During the degradation experiment, these samples were incubated at 37° C. on a shaker at 70 rpm. The remaining mass of each hydrogel was measured at time points 3, 6, 12, 18, 24, 36, and 48 hours. At each time point, the samples were washed with PBS three times to ensure the removal of the enzyme. The gels were then stored at −80° C. overnight before lyophilization to obtain the dry weights of the degraded gels. The dry weights of the gel samples were acquired after degradation. To calculate the percent mass remaining after degradation, the dry weight after degradation was divided by the initial weight of the hydrogel. The resulting ratios were converted to percentages.

### Mechanical Testing

The PCL-CaO₂ microparticle reinforced GelMA hydrogels used for mechanical analyses were prepared using the same method as described. As mentioned, the hydrogels were soaked in PBS for 24 hours to reach swelling equilibrium. An 8 mm biopsy punch was utilized to create uniform samples. The excess and residual liquid from the gels were removed using a Kimwipe. The compression test was conducted with a 0.0010 N preload force at an isothermal temperature of 23° C., soak time of 1 minute, force ramp rate of 0.1 N/min, and upper force limit set to 2 N. The linear region of the stress-strain curve was used to obtain the compressive modulus.

### Scanning Electron Microscopy (SEM) Imaging

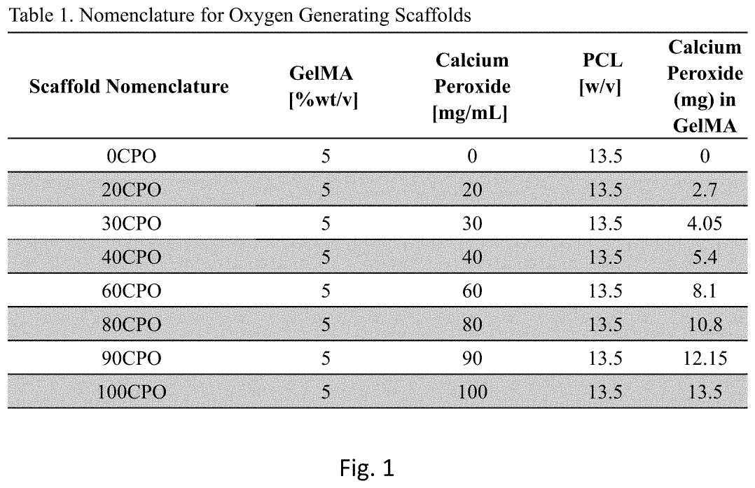

The porosity of each hydrogel sample was characterized using SEM imaging (JEOL 5200 SEM). These SEM images (FIG. 1) were also used to observe the morphology of the PCL-CaO₂ microparticles encapsulated within the entire scaffold. The hydrogel samples were flash frozen in liquid nitrogen, lyophilized, and coated with gold under argon atmosphere. The SEM images reveal the porous surface morphology of the oxygen generating microparticles and how they integrate within the GelMA hydrogel matrix.

### Oxygen Release Measurements

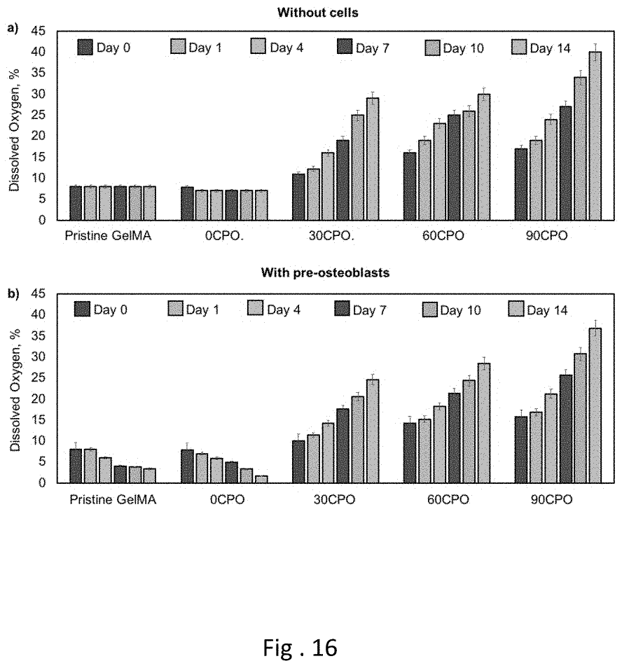

The samples were cultured under controlled hypoxic conditions in which the oxygen concentration was kept at 2% (StemCell Technologies Hypoxia Chamber) to study the oxygen release kinetics from the particles independent of atmospheric dissolved oxygen levels in media. The cells under hypoxia were limited to the oxygen from the microparticles as the primary oxygen source. Over a 14-day period, the change in the dissolved oxygen concentration was recorded for the samples that did not contain cells, and the samples that included preosteoblasts that were cultured in the oxygen-generating hydrogel scaffolds. The oxygen levels were monitored over time in the presence of catalase. Catalase is an enzyme that is produced by the liver and is known to increase the conversion efficiency of hydrogen peroxide to water and oxygen. Catalase was added into the cell culture media at 1 mg/mL concentration. The resulting oxygen release profile was then measured to study the effects of the oxygen content in the scaffolds on the change in oxygen levels in the culture media over 14-days. We utilized a handheld optical oxygen sensing probe (NeoFox) to measure the amount of oxygen (FIG. 16).

### Three-Dimensional (3D) Cell Encapsulation in Oxygen-Generating Scaffolds

In the cytocompatibility studies, preosteoblasts were encapsulated in the PCL-CaO₂ oxygen generating microparticle reinforced GelMA solutions at a cell seeding density of 5×10⁶ cells/mL. For 3D encapsulation, the preosteoblasts were trypsinized from the cell culture flasks, transferred into a falcon tube, and centrifuged to form a pellet. The number of cells were counted to determine the appropriate amount of cells to be transferred and resuspended in the oxygen-generating prepolymer solution. Subsequently, 40 μL of the prepolymer solution containing the preosteoblasts were pipetted in the wells of a 96-well plate. The hydrogels were photocrosslinked at 700 mW/cm². Then, over a period of 2 weeks, the microencapsulated preosteoblast-laden oxygen-generating gels were cultured under hypoxic conditions. The samples were then analyzed for their oxygen content, mechanical properties, proliferation, cytotoxicity, and apoptosis.

### Cell Proliferation, Cytotoxicity, Apoptosis, and ALP Activity

The cells were resuspended in the 5% (w/v) GelMA prepolymer along with 0, 30, 60, or 90 mg/mL CaO₂ in PCL microparticles. The preosteoblast-laden scaffolds were cultured in a DMEM medium supplemented with 10% (v/v) fetal bovine serum (FBS) and 5% (v/v) penicillin/streptomycin. The cells were maintained in a 37° C. incubator with 5% carbon dioxide (CO₂) and the culture media was replaced every 2-3 days. These preosteoblasts were then assessed for proliferation using the Alamar Blue assay. Following 4 h of incubation in the Alamar Blue solution, the fluorescence of the supernatant from the samples was read using a microplate reader. The fluorescence values of the resulting solutions were recorded at 560 nm/590 nm (Ex/Em).

A commercially available lactate dehydrogenase (LDH) (Invitrogen) assay was performed to evaluate the cytotoxic effects of the oxygen-generating scaffolds. The released LDH acts as a catalyst in the conversion of lactate to pyruvate and NAD+ reduction to NADH. The resultant diaphorase then utilizes the NADH to reduce a tetrazolium salt (INT) to a red formazan. The fluorescence of formazan is directly proportional to the LDH released by the cells in the culture media²⁷. To conduct this experiment, a 25 μL of the sample was pipetted into a 96-well plate and allowed to react with 25 μL of the reaction analyte for 30 minutes. A stop solution was then added to the mixture, and the absorbance was read at a wavelength of 490 nm. Four replicates were performed for each scaffold composition. Furthermore, a Caspase Glo 3/7 assay (Promega) was performed to evaluate whether cellular apoptosis occurred. Protected from light, the Caspase Glo 3/7 reaction substrate was added to the samples in a 1:1 ratio and allowed to react for 45 minutes. A stop solution was added after 40 minutes and the luminescence of the resulting solution was recorded.

The pH of the cell culture solution can also be a contributing factor to viability and metabolic activity of cells in tissue-engineering construct. The effect of the oxygen-generating scaffolds on the pH of the cell culture media was measured over a 14-day in vitro experiment. For this analysis, commercial pH strips were used to measure the pH.

### Gene Expression

The total RNA was extracted from the oxygen-generating scaffolds after day 14 of culture period using the RNAqueous Kit (Invitrogen) according to the manufacturer's protocol. A Nanodrop2000 system was used to evaluate the quality and quantity of the RNA. Then, RT-qPCR was performed using the RNAqueous Kit the Verso One-Step RT-qPCR Kit, SYBR Green and Low ROX (Thermo Fisher) with CFX Connect Real-Time System (Bio-Rad) according to the manufacturer's protocol. The melting curves were evaluated for the samples. The target gene expressions were normalized to housekeeping gene (GAPDH) expression levels. The primers designed were: BMP-7 (forward, 5′-TACATGGGAAAC CTGGGTAAAG-3; reverse, 5′-GGTGACATTCTGTCGGGTAAA-3′), osteocalcin (forward, 5′-TGTGTCCTCCTGGTTCATTTC-3; reverse, 5′-CTGTCTCCCTCATGTGTTGTC-3′), and GAPDH (forward, 5′-CGCCCTGATCTGAGGTTAAAT-3; reverse, 5′-CGGAGCAACAGATGTGTGTA-3′).

### Statistical Analysis

GraphPad Prism 6.0 (La Jolla, Calif., USA) was used for the statistical analyses. The results were assessed by performing a one-way ANOVA. Bonferroni post hoc tests were carried out to analyze the statistically significant differences. A p value <0.05 was considered to be a statistically significant difference in all shown analyses. All values are represented as averages±standard deviation (*p<0.05, **p<0.01, ***p<0.001, and ****p<0.0001).

### Characterization of the Oxygen-Generating Scaffolds

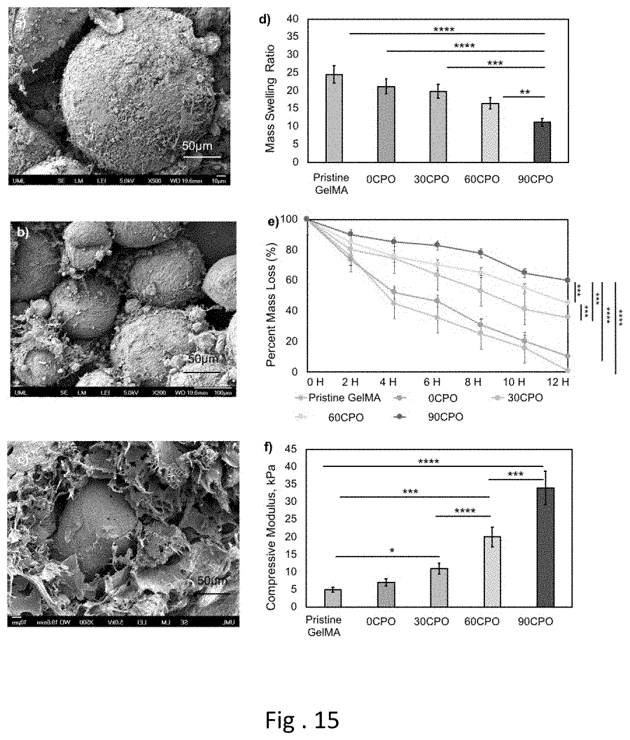

The synthesized oxygen-generating scaffolds were characterized to reveal the physical properties of the resulting materials such as swelling, degradation, mechanical strength, and morphology. FIG. 15 shows the biomaterial properties as well as the morphology of the PCL-CaO₂ microparticles that were synthesized according to the previously described protocol. The average size of the oxygen generating microparticles was 100 μm. The SEM imaging captured the topography of the oxygen-generating microparticles and their integration with the hydrogel matrix. These microparticles were encapsulated with the hydrogel matrix at a concentration of 13.5% (w/v) to fabricate the oxygen-generating scaffolds. The swelling ratios shown in FIG. 1 were calculated by measuring the wet weight and dry weights of the scaffolds as described in the previous sections. As expected, the swelling ratios demonstrated a decreasing trend with the increasing concentrations of CaO₂ in PCL. Specifically, the results show that the swelling ratios were 25%, 21%, 20%, 17%, and 11% for the Pristine GelMA, 0CPO, 30CPO, 60CPO, and 90CPO scaffolds, respectively. The degradation tests show that at the end of the 48-hour degradation experiment there was 1%, 10%, 36%, 45%, and 60% of the scaffold mass remaining for the pristine GelMA, 0CPO, 30CPO, 60CPO, and 90CPO groups, respectively. Therefore, the higher concentrations of CaO₂ show decreased rate of degradation of the scaffolds. The mechanical properties of the oxygen-generating scaffolds were evaluated using a DMA compression analysis. The DMA analysis revealed a compressive modulus of 5±0.81 kPa, 7±0.77 kPa, 11±0.8 kPa, 20±0.69 kPa, and 34±0.9 kPa for the Pristine GelMA, 0CPO, 30CPO, 60CPO, and 90CPO scaffolds, respectively. As shown, the compressive strength is correlated to amount of CaO₂ content in the microparticles. There was increasing trend in compressive moduli with increasing concentration of the CaO₂ in PCL microparticles, as expected.

### Oxygen-Release Kinetics

The oxygen-release profiles of the oxygen-generating scaffolds were evaluated through the percentage of dissolved oxygen in the cell culture media. The effects of differing amounts of CaO₂ in the composite scaffolds on the oxygen-release kinetics were studied with and without preosteoblasts in the presence of 1 mg/mL catalase the media. These experiments indicated that there were observable differences across different experimental conditions with and without cells encapsulated in the oxygen-generating scaffolds.

In the scaffolds cultured without cells, there was a peak of 29%, 30%, and 40% dissolved oxygen by day 14 for the 30CPO, 60CPO, and 90CPO groups, respectively. In contrast, the pristine GelMA and 0CPO groups possessed a maximum dissolved oxygen on day 0, 8% and 7%, respectively. The scaffolds with 3D-encapsulated preosteoblasts demonstrated oxygen-release profiles similar to the scaffolds without cells. The peak for dissolved oxygen was demonstrated on day 0 for pristine GelMA and 0CPO, as anticipated because the amount of oxygen decreased over time due to the hypoxia conditions. There was significant decrease in this measurement on day 14, indicating 3% and 2% dissolved oxygen for GelMA and 0CPO, respectively. There was significantly increased oxygen content in these conditions shown in the 30CPO, 60CPO, and 90CPO with 25%, 28%, and 37% dissolved oxygen, respectively on day 14.

### Cell Proliferation, Cytotoxicity, Apoptosis, and ALP Activity

### Alamar Blue Activity of Preosteoblasts in Oxygen-Generating Scaffolds

The Alamar Blue assay assessed the metabolic activity of the preosteoblasts encapsulated in the oxygen-generating scaffolds. In particular, the results showed that the cellular metabolic activity increased by different magnitudes across all scaffolds cultured under hypoxia and in the presence of catalase in the media. The Pristine GelMA and 0CPO scaffolds showed the least metabolic activity by day 7 and followed by a considerable decrease in metabolic activity on day 14. However, the 30CPO scaffolds showed improved metabolic activity which eventually decreased on day 14. The 60CPO group showed a significantly highest metabolic activity on days 0, 1, 4, 5, 10 and 14, with respect to the Pristine GelMA and 0CPO control groups as well as the 90CPO scaffolds. Whereas the 90CPO scaffolds showed decreased metabolic activity by day 14.

### LDH Activity of Preosteoblasts in Oxygen-Generating Scaffolds

The lactate dehydrogenase (LDH) activity of the 3D encapsulated preosteoblasts in oxygen-generating scaffolds was evaluated using an LDH cytotoxicity assay according to the standard manufacturer's protocol. LDH is an enzyme found in living cells and facilitates the conversion of lactate to pyruvate and vice versa. The LDH is released during cellular damage, and can therefore, be used as a marker to assess cytotoxicity²⁸. The LDH assay was performed for the cells cultured in the oxygen-generating scaffolds using the manufacturer's protocol. The results indicated that the LDH activity showed an observable increase from day 1 through day 14. The 30CPO scaffolds showed a lower gradual increase in the LDH activity than the pristine GelMA and 0CPO scaffolds. However, the 60CPO scaffolds showed no significant increase in the LDH activity on day 14 compared to day 0. In addition, the 60CPO scaffolds reported the lowest LDH activity among all scaffold compositions pointing out the least amount of cellular damage compared to the rest of the experimental conditions. At the highest CaO₂-PCL concentration, the 90CPO scaffolds showed a sharp significant increase in the LDH activity, an indication of oxidative damage.

### Caspase Glo 3/7 Activity of Pre-Osteoblasts in Oxygen Generating Scaffolds

The Caspase Glo 3/7 assay was used to evaluate the apoptosis of preosteoblasts within the oxygen-generating scaffolds. In this assay, the caspase cleaves off the caspase-3/7 DEVD-aminoluciferin substrate reaction substrate, resulting in the release of aminoluciferin which is then consumed by luciferase. Subsequently, this protein-protein interaction generates a luminescence signal²⁹. This luminescence signal is proportional to the Caspase 3/7 activity which indicates the presence of cellular apoptosis. We report that the pristine GelMA and 0CPO scaffolds showed a sharp increase in the Caspase 3/7 activity. Contrarily, the 30CPO scaffolds showed a lower increase in Caspase 3/7 activity in comparison to the pristine GelMA and 0CPO scaffolds. The 90CPO group showed the highest increase in Caspase 3/7 activity across all scaffold groups. As the optimal condition, the 60CPO scaffolds showed the least amount of increase of Caspase 3/7 activity during this experiment.

### ALP Activity of Preosteoblasts in Oxygen-Generating Scaffolds

The alkaline phosphatase (ALP) is an early marker to evaluate osteogenic differentiation^{30,31}. The ALP activity was measured for the cells that were encapsulated within the oxygen-generating scaffolds. The ALP activity increased by day 14 across all groups; however, the 30CPO and 60CPO scaffolds demonstrated significantly higher ALP activity on day 14 with respect to the pristine GelMA and 0CPO groups. In contrast, the 90CPO group showed the lowest ALP activity on day 14. These results were achieved without any osteogenic supplements in the media to evaluate the osteoinductivity of the scaffolds with different oxygen content. In particular, the 60CPO scaffolds exhibited the strongest osteoinductive behavior. Therefore, the presence of the oxygen-generating scaffolds provided a microenvironment conducive to osteogenic differentiation under hypoxia.

### pH Measurements

The effect of the oxygen-generating scaffolds on the pH of the culture media was measured using pH strips on day 14 of the in vitro study. Four replicates were performed for each scaffold composition. The results showed no significant differences in the pH measurements for the pristine GelMA, 0CPO, 30CPO, 60CPO, and 90CPO scaffolds. The pH measured for the pristine GelMA, 0CPO, 30CPO, 60CPO, and the 90CPO scaffolds were pH 8, 8.5, 8.5, 9, and 9, respectively.

### Gene Expression

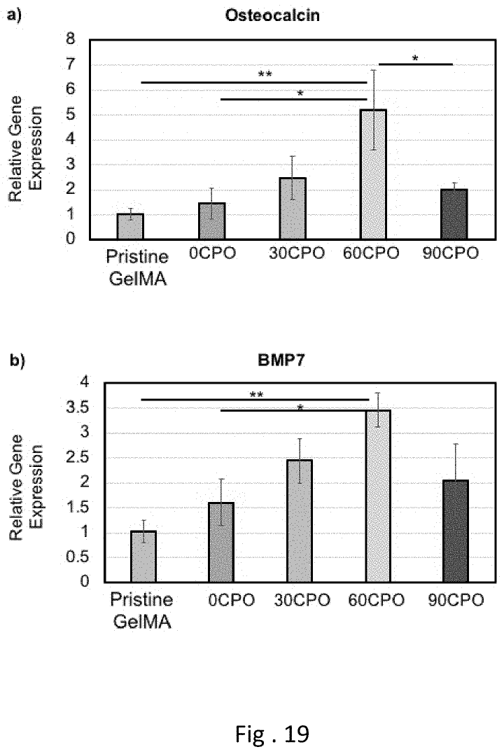

A RT-qPCR technique was performed using the messenger RNA (mRNA) isolated from the preosteoblasts in the oxygen-generating scaffolds on day 14. The mRNA was probed for late differentiation markers in the preosteoblasts. The two differentiation markers included the bone morphogenic protein 7 (BMP 7) and the osteocalcin (OCN). BMP and OCN are commonly analyzed genes to study the osteogenic differentiation of preosteoblasts. FIG. 19 revealed how the BMP-7 and OCN genes expressed by the preosteoblasts after 14 days cultured in the oxygen-generating scaffolds. The BMP-7 mRNA expression in the PCL-CaO₂ composite scaffolds was significantly higher for the 60CPO than those of the pristine GelMA, 0CPO, 30CPO, and 90CPO conditions (p<0.001). The 30CPO scaffolds showed higher mRNA expression than the pristine GelMA and 0CPO scaffolds but lower than the 60CPO groups. Interestingly, the 90CPO scaffolds showed low BMP-7 and OCN expression levels comparable to the pristine GelMA and the 0CPO groups. This result, therefore, suggested that the 60CPO scaffolds significantly improved osteogenic differentiation of the encapsulated preosteoblasts. Overall, there was a range of 30-60 mg/mL CaO₂ in PCL to potentially support and induce differentiation of preosteoblasts into mature osteoblasts.

### Discussion

### Characterization of the Oxygen-Generating Scaffolds

### Synthesis of the Oxygen-Generating Scaffolds and Characterization of the Mechanical Properties

The ratio of the oxygen-generating microparticles within the hydrogel matrix is critical to ensure that the appropriate partial pressure of oxygen is delivered and maintained in the extracellular microenvironment. Through our in vitro experiments, we optimized a system where a mean microparticle size of 100 μm was found to effectively provide sustained oxygen release at a 13.5% (w/v) concentration in the GelMA prepolymer matrix. A cell density of 5×10⁶ cells/mL was used for preosteoblast encapsulation in the scaffolds to standardize our system. The only variable in our system was the concentration of CaO₂ within the PCL. The results suggested that oxygen generation was dependent on amount of CaO₂ content in matrix. The other factors that could affect oxygen generation may include the crosslinking density, material stiffness, pore size, and porosity of the biomaterial, which were all kept standard for our scaffolds to ensure only one parameter variation. These properties also inherently influence mechanical properties such as swelling, degradation and compressive strength. The physical properties are also important at affecting cellular behaviors such as cell spreading, viability, and proliferation. The manner in which the 3D encapsulated cells interact with each other dictate the mechanotransduction experienced by the cells in the microenvironment, which in turn, guides their development and differentiation. The stiffness of the hydrogel matrix is critical for guiding cell differentiation and proliferation in 3D scaffolds and has been shown to be substantially affect the pore size, structure, porosity and interconnectivity of the pores which all play a crucial role in guiding cell differentiation and proliferation. To evaluate and quantify these effects of the hydrogel matrix stiffness, the DMA analysis had been used to determine the compressive moduli of the oxygen-generating scaffolds. Our results showed that the addition of the microparticles progressively improved the mechanical behavior of the scaffolds with an increase in the CaO₂ concentration within our scaffolds. Our results show that specifically, the modification of the CaO₂ content in the oxygen-generating microparticles can be used to modulate the mechanical strength. Therefore, the compressive moduli increased with the increase in the concentration of CaO₂. The control of the mechanical properties also improved the osteogenic differentiation of the 3D-encapsulated preosteoblasts.

### Swelling Behavior of Oxygen-Generating Scaffolds

The swelling capacity and behavior of the hydrogel matrix affects the material's porosity, stiffness, and structural stability. The swelling properties of the oxygen-generating hydrogel scaffolds were studied in varying CaO₂ concentrations within the PCL and control groups. Based on the findings, we report that the swelling behavior can be tuned by modulating the concentrations of the oxygen-generating compound CaO₂. The pristine GelMA scaffolds offers higher hydrophilic scaffold matrix as there is no presence of microparticles within the matrix and swell the most. Without any oxygen-generating content, there was significantly higher swelling ratio as compared to the other groups that included different concentrations of CaO₂ in the PCL (p<0.001). The swelling behavior decreased accordingly based on the amount of CaO₂ in the microparticle reinforcement. As hypothesized, the scaffold composed of the highest CaO₂ content (i.e. 90CPO) presented the lowest swelling ratio as compared to the other scaffold compositions (p<0.001). With more CaO₂ in the oxygen-generating microparticles, the amount of the gel is less in the composite scaffolds resulting in lower swelling ratios. This result is expected because a decrease in the hydrophilic content causes a reduction in the swelling capacity.

### Degradation Behavior of Oxygen-Generating Scaffolds

As the 3D scaffolds degrade, the cells encapsulated within the scaffolds are exposed to void sites in the native tissue to deposit their own ECM, which in turn facilitates the formation of the new tissue³⁶. Therefore, the degradation behavior of the oxygen-generating scaffolds was characterized to understand their regenerative potential for new tissue formation and predict the success of the implants in vivo. Ideally, a bone scaffold should possess degradation behavior that is similar to the rate of bone regeneration and formation. In this experiment, the enzymatic degradation behavior of the PCL-CaO₂ reinforced hydrogels was determined in vitro via an accelerated enzymatic degradation approach. Collagenase II is an enzyme that degrades collagen and serves to degrade the polymeric structure of the GelMA hydrogel matrix. This enzyme was overall effective in degrading the hydrogel component of the oxygen-generating scaffolds. According to the results, the degradation rate of the composite hydrogel scaffolds decreased with an increase in the concentration of CaO₂ in the PCL. We attribute this biodegradation behavior to the increase in the CaO₂ content within the microparticle and scaffold. With increased CaO₂, there is greater non-hydrogel component occupying more volume within the scaffold matrix As a result, the % mass remaining reported are the scaffold components that do not undergo enzymatic degradation (i.e. PCL-CaO₂ content) in the matrix. This rationale also supports the highest degradation found in the pristine GelMA which did not contain the PCL-CaO₂ microparticles. This condition showed the highest degradation and therefore the lowest % mass remaining post degradation. Our results demonstrated that different concentrations of CaO₂ in the composite hydrogels significantly influenced the degradation behavior. The findings offer a clear demonstration that PCL-CaO₂ microparticle reinforced hydrogel scaffolds show highly tunable degradation properties.

### Microarchitecture of the Oxygen-Generating Scaffolds

The high-resolution SEM imaging of the oxygen-generating scaffolds captured microparticle morphology, distribution, pore structure, and interaction with the hydrogel matrix (FIG. 15). The majority of the PCL-CaO₂ microparticles were within 100 μm mean diameter. The average diameter of the oxygen-generating microparticles was 100 μm as confirmed from the SEM imaging which was kept standard throughout all the experiments. The surface morphology of the PCL-CaO₂ microparticles was porous and rough. The cross-section image of the Pristine GelMA scaffold groups indicated highly porous interconnected lace-like matrix. The porous interconnected matrix morphology of the hydrogels allowed for cell migration and infiltration in 3D. The SEM images of the PCL-CaO₂ reinforced hydrogels also revealed a highly porous macrostructure with good pore interconnectivity. Based on the morphology shown, the surface of the PCL-CaO₂ microparticles indicated integration within the hydrogel matrix. The presence of lace-like structure over the microparticles in particular showed integration of the microparticles within the crosslinked GelMA polymer. This microarchitecture of oxygen-generating scaffolds offers a 3D structure to house cells and facilitate cell infiltration and migration similar to the pristine GelMA control group.

### Oxygen-Release Kinetics

The oxygen-release kinetics were observed in diverse culture conditions including with and without cells, under induced hypoxia, and the addition of catalase. The preosteoblasts at 5×10⁶ cells/mL cell seeding density were 3D encapsulated with the oxygen-generating microparticles at 13.5% (w/v) in GelMA. The results indicated that the dissolved oxygen increased over time with an increase in the concentration of CaO₂ in PCL, as expected. The scaffolds without 3D encapsulated preosteoblasts, demonstrated an increasing trend in the dissolved oxygen in the media with no cells in the scaffold matrix to consume the released oxygen. The dissolved oxygen content in the pristine GelMA, 0CPO, 30CPO, 60CPO, and 90CPO scaffolds continued to increase and then reached to a peak value. After the peak release of the oxygen, the oxygen measurement plateaued briefly before decreasing gradually over the 14 day period. As hypothesized, the oxygen release is higher, when the concentration of CaO₂ in PCL is higher, as there is more solid peroxide content to react with the surrounding water content. On the other hand, the Pristine GelMA and 0CPO groups have no oxygen-generating content (i.e. CaO₂) present. Therefore, there was no significant change in the dissolved oxygen content in these scaffold compositions. This decreasing trend with cells was also expected as the cells were limited to the oxygen that is released by the scaffolds. Therefore, without the oxygen-releasing microparticles, there is a reduced amount of dissolved oxygen (%) in media over time under hypoxia. The pristine GelMA and 0CPO scaffolds with cells showed that amount of dissolved oxygen (%) levels decreased steadily and then reached the hypoxic equilibrium 2% dissolved oxygen over the rest of the 14 day in vitro culture period.

### Cell Proliferation, Cytotoxicity, Apoptosis, and ALP Activity

### Metabolic Activity of Oxygen-Generating Hydrogels

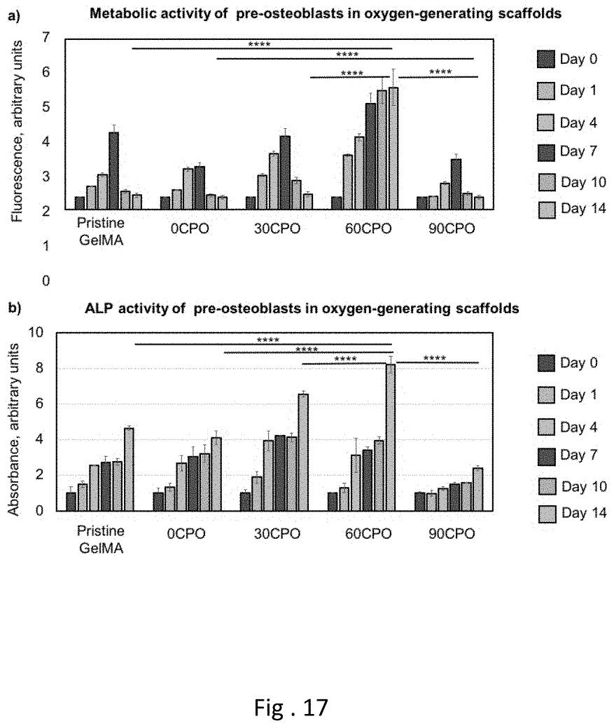

The metabolic activity of the encapsulated preosteoblasts in various scaffold compositions of PCL-CaO₂ reinforced hydrogels was assessed on days 1, 4, 7, 10 and 14 in cell culture under hypoxic conditions with the addition of 1 mg/mL catalase in the culture media (FIG. 17 a). The metabolic activity of preosteoblasts increase over the course of 7 days for all the scaffold compositions. However, the 60CPO scaffolds show the highest metabolic activity across all time points among the other test conditions (i.e. Pristine GelMA, 0CPO, 30CPO, and 90CPO scaffolds). While the 30CPO scaffolds do not show exceptional metabolic activity, the presence of the CaO₂ in PCL at the 30 mg/mL concentration within PCL does show improved metabolic activity as compared to the Pristine GelMA and 0CPO groups. Conversely, at the highest concentration, the 90CPO group on the other hand showed a decreasing level of metabolic activity. This metabolic activity drop is indicative of oxidative damage due to excessive oxygen in the cellular microenvironment. The findings suggest there is a favorable range of CaO₂ concentrations within PCL, between the 30CPO and 60CPO compositions that supports optimal metabolic function of the 3D encapsulated preosteoblast. Here, the engineered system that offered superior biological performance included a cell seeding density of 5×10⁶ cells/mL and the 60CPO scaffold. This scaffold composition offers the exceptional metabolic response and supports the cell survival and function, especially under hypoxia.

### LDH Assay for Evaluation of Cytotoxicity

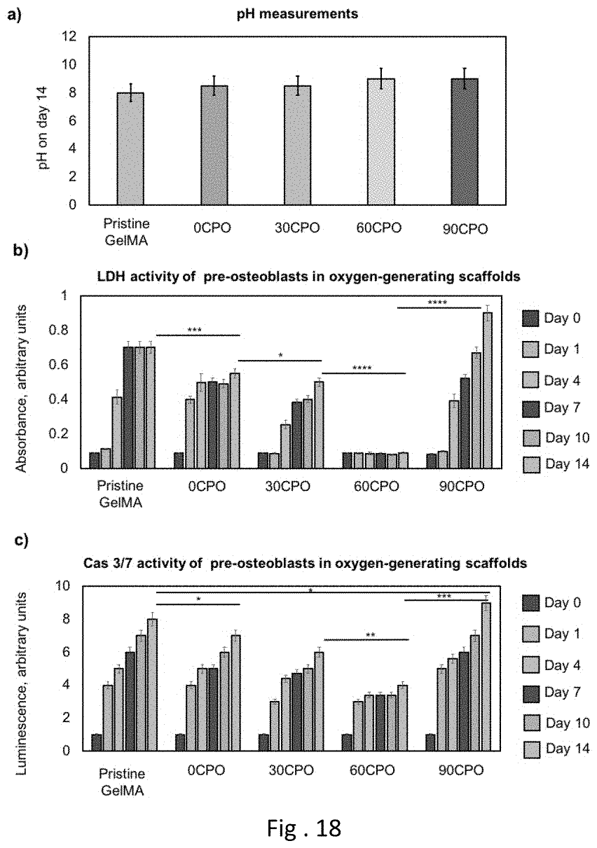

The LDH assay demonstrated the cytotoxic effects of oxygen-regenerating scaffolds at extreme CaO₂ concentrations in PCL. When cultured under hypoxia and with catalase, the LDH activity increased overtime for the Pristine GelMA, 0CPO, 30CPO and 90CPO scaffolds (FIG. 18b). This increase in LDH activity overtime for the Pristine GelMA and 0CPO scaffolds was attributed to hypoxia-induced necrosis as these scaffolds were devoid of CaO₂ and therefore deprived any component that would provide oxygen supply. These findings are consistent with our Alamar blue assay results which revealed low cell viability in these scaffolds. The 30CPO was also consistent with other assays that show that the scaffold composition is not entirely sufficient to support cell viability, but it is an improved version of its control. As discussed, at extreme conditions (i.e. 90CPO), there is an increase in the LDH activity over time which is indicative of oxidative damage due to excess oxygen. While oxygen supply is necessary, the excessive amounts of oxygen in the cellular microenvironment damages the 3D encapsulated cells. The 60CPO scaffolds showed constant LDH levels with no significant changes in LDH levels overtime which indicate that the 60CPO scaffolds were most favorable and did not elicit any cytotoxicity.

### Evaluation of Apoptosis of Osteoblasts

The Caspase-Glo® 3/7 Assay (Promega) is a luminescence assay that measures caspase-3 and -7 enzymatic activity. The assay substrate is a luminogenic caspase-3/7 complex, which consists of the DEVD tetrapeptide sequence. This reagent is optimized for caspase activity, luciferase activity, and cell lysis. The addition the Caspase-Glo® 3/7 reagent results in cell lysis and the caspase cleavage of the reaction substrate. This cleavage of the DEVD substrate produces release of energy in the form of a luminescence signal which is shown in FIG. 4. The generated luminescence is proportional to the amount of caspase 3 and 7 activity, which is expressed during cellular apoptosis. The Caspase 3/7 results demonstrated an increase in caspase activity overtime for the Pristine GelMA and 0CPO scaffolds. These results are attributed to the absence of CaO₂ in both groups which results in long-term hypoxia-induced damage to the 3D encapsulated cells. The 30CPO scaffolds also exhibited an increase in the caspase activity but is lower than the Pristine GelMA and 0CPO scaffolds. Therefore, the presence of 30CPO bone scaffold improves cellular response; however, it is not sufficient for long term cytocompatibility. In contrast, the 90CPO scaffolds presented a considerable increase in caspase activity overtime which is indication of oxidative damage to the preosteoblasts. This oxidative damage is likely due to the presence of excessive oxygen in the cellular microenvironment. Contrarily, the 60CPO scaffolds showed no increase in the caspase activity overtime indicating that the 60CPO scaffolds. Therefore, the 60CPO offers a moderate and optimal scaffolding condition for supporting cell viability with minimal damaging effects.

### Alkaline Phosphatase (ALP) Activity of Preosteoblasts in Oxygen-Generating Scaffolds

Alkaline phosphatase (ALP) is an early differentiation marker for osteoblast precursor cells. From the 14 day in vitro study, the ALP values for the oxygen-generating scaffold conditions were normalized to day 1 (FIG. 17b). These results support that the ALP activity of the encapsulated preosteoblasts increased overtime from day 1 through day 14. Moreover, across all scaffold conditions the highest level of ALP is found in day 14. As there no oxygen-releasing content, the Pristine GelMA and 0CPO groups showed the lowest ALP activity and resulted in the cells undergoing necrosis. The 30CPO scaffolds demonstrated higher ALP activity as compared to the negative control groups. This behavior suggests that the presence of CaO₂ within the PCL helps improve cell viability and early differentiation. As shown, the optimal condition the 60CPO scaffolds showed the highest increase in ALP activity overtime and maintain the highest ALP levels measured across all conditions. The 90CPO scaffolds showed the lowest ALP activity which we attribute to the oxidative damage and potential cytotoxic effects caused to the cells due to excessive dissolved oxygen in media. The findings of this assay are also consistent with our findings of other in vitro functional assays.

### Osteogenic Gene Expression in Oxygen-Generating Scaffolds

The RT-qPCR analysis of the mRNA isolated from the encapsulated preosteoblasts demonstrate the osteoinductive performance of the PCL-CaO₂ microparticle reinforced scaffolds. The mRNA was quantified, and their expression levels were mapped for late differentiation markers for the osteoblasts. The results of studying gene expression revealed that the 60CPO scaffolds showed significantly higher expression levels in comparison to scaffolds with lower and higher CaO₂ content. Therefore, the results suggest 60CPO scaffolds facilitate superior osteogenic differentiation to other the conditions tested. Furthermore, the analysis also suggests there is a critical range of dissolved oxygen concentration in the cellular microenvironment required for these processes to occur.

### Conclusion

Oxygen plays a critical role in maintaining cell viability and function of metabolically active cells that are encapsulated in engineered tissue constructs. In this work, calcium peroxide was encapsulated in PCL as a hydrophobic barrier to form composite oxygen-generating microparticles using an emulsification approach. The oxygen-generating microparticles were used to reinforce gelatin-based hydrogel for generation of the oxygen-generating scaffolds. The hydrophobic nature of the PCL reduces the rapid hydrolysis that occurs when the water in the hydrogel matrix reacts with the encapsulated CaO₂. The ability to provide a controlled and sustained oxygen release benefits cells that are encapsulated within scaffold in 3D. Our results revealed the capacity of these scaffolds to support viability, proliferation, and cytocompatibility of preosteoblasts. The scaffolds with different contents of oxygen yielded in distinct oxygen-release profiles. The increasing concentrations of CaO₂ within the PCL provided increasing amounts of oxygen release in these scaffolds. The oxygen release kinetics were monitored over the 14 days in the absence and presence of preosteoblasts. The 60CPO scaffolds showed an optimal oxygen release rate and peak release potential to support the survival and proliferation of the encapsulated preosteoblasts. The biological analysis of the 3D encapsulated preosteoblasts demonstrated that the 60CPO scaffolds supported cell growth and proliferation most optimally under the hypoxic culture conditions. Further analysis demonstrated low levels of LDH and Caspase 3/7 for the 60CPO scaffolds, indicating that the 60CPO scaffolds did not cause significant cellular damage but highly supported cell proliferation. The ALP measurements and gene expression analyses also indicated the 60CPO scaffolds presented exceptional osteoinductivity for the encapsulated preosteoblasts. The high tunability and precise control of release kinetics of oxygen in these composite scaffolds are anticipated to support engineering off-the-shelf bone tissue constructs in future applications.

Therefore, it can be seen that the present invention provides a unique solution to the problem of providing an oxygen releasing biomaterial that overcomes the disadvantages of the prior art by providing an oxygen releasing biomaterial that resists burst release of oxygen and has a sustained and gradual release of oxygen over a period of four to five weeks. The oxygen releasing biomaterial has wide application in medical treatment, including tissue engineering scaffolds, cardiac conditions, osteogenesis, wound treatment, including burns, and antimicrobial properties. Accordingly, the oxygen releasing biomaterials represent a significant improvement over prior art biomaterials.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. An oxygen-releasing biomaterial, comprising:
a hydrogel; and
a plurality of microparticles suspended in the hydrogel, the microparticles comprising an oxygen carrier encapsulated in a hydrophobic material, the hydrophobic material comprising a biocompatible polymer;
wherein the oxygen carrier has a sustained release of oxygen from the hydrophobic material over a five-week period.

2. The biomaterial of claim 1 wherein the oxygen carrier comprises solid peroxides, liquid peroxides, or fluorocarbons.

3. The biomaterial of claim 2, wherein the oxygen carrier comprises CaO₂.

4. The biomaterial of claim 1, wherein the biocompatible polymer is a biodegradable polyester.

5. The biomaterial of claim 3, wherein the biocompatible polymer is selected from the group consisting of poly dimethyl siloxane, polylactic acid (PLA), polyglycolic acid (PGA), polylactic co-glycolic acid (PLGA), polyhydroxybutyrate (PHB), poly(3-hydroxy valerate), poly(ethylene succinate) (PESu), poly(butylene adipate-co-terephthalate) (PBAT), Poly(glycerol sebacate) (PGS), polyhydroxyalkanoates (PHAs), polyurethanes, poly vinyl pyrrolidone, or polycaprolactone (PCL).

6. The biomaterial of claim 1, wherein the plurality of microparticles are sized about 50 μm to about 250 μm in diameter.

7. The biomaterial of claim 1, wherein the plurality of microparticles comprises from about 20 mg/mL to about 100 mg/mL of CaO₂ within PCL encapsulated within the hydrogel suspension, equivalent to 2.7 mg-13.5 mg CaO₂ per mL of GelMA hydrogel precursor solution.

8. The biomaterial of claim 1, wherein the plurality of microparticles comprises about 5-25% w/v of the hydrogel suspension.

9. The biomaterial of claim 1, wherein the hydrogel is selected from the group consisting of synthetic and natural polymers including polyvinyl alcohol, polyethylene glycol, sodium polyacrylate, acrylate polymers and copolymers with an abundance of hydrophilic groups, alginate, agarose, gellan gum, guar gum, dextran, heparin, chondroitin sulfate, dermatan sulfate, hyaluronic acid, and proteins including collagen, gelatin, elastin, laminin, and fibrin, and combinations thereof.

10. The biomaterial of claim 9, wherein the hydrogel comprises gelatin methacrylate.

11. The biomaterial of claim 1, further comprising a photoinitiator.

12. An oxygen-releasing biomaterial, comprising:
a hydrogel; and
a plurality of microparticles suspended in the hydrogel, the microparticles comprising an oxygen carrier comprising a solid peroxide encapsulated in a hydrophobic material, the hydrophobic material comprising a biocompatible polymer, the plurality of microparticles comprising 5-25% w/v of the hydrogel suspension;
wherein the oxygen carrier has a sustained release of oxygen from the hydrophobic material up to a five-week period.

13. The biomaterial of claim 12, wherein the oxygen carrier comprises CaO₂.

14. The biomaterial of claim 12, wherein the biocompatible polymer is polycaprolactone (PCL).

15. The biomaterial of claim 12, wherein the plurality of microparticles are sized about 50 μm to about 250 μm in diameter.

16. The biomaterial of claim 12, wherein the plurality of microparticles comprises from about 20 mg/mL to about 100 mg/mL CaO₂ in PCL at about 13.5% w/v of the GelMA hydrogel precursor.

17. The biomaterial of claim 12, wherein the hydrogel is selected from the group consisting of synthetic and natural polymers including polyvinyl alcohol, polyethylene glycol, sodium polyacrylate, acrylate polymers and copolymers with an abundance of hydrophilic groups, alginate, agarose, gellan gum, guar gum, dextran, heparin, chondroitin sulfate, dermatan sulfate, hyaluronic acid, and proteins including collagen, gelatin, elastin, laminin, and fibrin, and combinations thereof.

18. The biomaterial of claim 17, wherein the hydrogel comprises gelatin methacrylate (GelMA).

19. The biomaterial of claim 12, further comprising a photoinitiator.

20. An oxygen-releasing biomaterial, comprising:
a hydrogel; and
a plurality of microparticles suspended in the hydrogel, the microparticles comprising an oxygen carrier comprising calcium peroxide encapsulated in a hydrophobic material, the hydrophobic material comprising polycaprolactone (PCL), the plurality of microparticles comprising 13.5% w/v of the hydrogel suspension;
wherein the microparticles are sized about 50 μm to about 250 μm in diameter;
wherein the oxygen carrier has a sustained release of oxygen from the hydrophobic material over a five-week period.
